(19) United States
(12) Patent Application Publication
(10) Pub. No.: US 2025/0381537 A1
(43) Pub. Date: Dec. 18, 2025

(54) LOW SHEAR TOROIDAL IMPELLER

(71) Applicant: Donaldson Company, Inc., Bloomington, MN (US)

(72) Inventors: Jacob Q. Fabro, Woodbury, MN (US); Jean-Christophe Drugmand, Wavre (BE)

(73) Assignee: Donaldson Company, Inc., Bloomington, MN (US)

(21) Appl. No.: 19/238,140

(22) Filed: Jun. 13, 2025

Related U.S. Application Data
(60) Provisional application No. 63/660,043, filed on Jun. 14, 2024.
(60) Provisional application No. 63/706,429, filed on Oct. 11, 2024.

(51) Int. Cl. B01F 27/91; B01F 27/1111; B01F 27/113; B01F 101/44

(52) U.S. Cl. CPC B01F 27/91; B01F 27/1111; B01F 27/113; B01F 2101/44

(57) ABSTRACT

A bioreactor can include a vessel defining an interior volume; a motor; a rotatable shaft assembly connected to the motor and extending within the interior volume along a longitudinal axis; one or more toroidal impellers mounted onto the shaft assembly and disposed within the interior volume, the toroidal impeller including a plurality of blade members supported by a hub that each define a radially bounded passageway extending along a second axis disposed at an oblique angle to the longitudinal axis.

### CROSS-REFERENCE TO RELATED APPLICATIONS

This application claims the benefit of U.S. Provisional Application Ser. No. 63/706,429, filed on Oct. 11, 2024; and U.S. Provisional Application Ser. No. 63/660,043, filed on Jun. 14, 2024, the disclosures of which are hereby incorporated by reference in their entireties.

### TECHNICAL FIELD

The present invention relates to impeller designs for use within mixing tanks, for example, blades used in low shear bioreactor mixing applications.

### BACKGROUND

A bioreactor typically consists of a tank or vessel designed to provide a suitable environment for biological processes. One aspect of bioreactor operation is efficient mixing, which ensures uniform distribution of nutrients, gases, and temperature throughout the culture medium in addition to efficient control of process parameters such as temperature, oxygen, CO₂, and pH levels. In many implementations, impellers provide for such mixing. Impellers are rotating devices within the bioreactor tank that generate turbulence and induce fluid circulation. Some known types of impellers for use in such applications are, with reference to FIGS. 28 to 31, typically Rushton type impellers 100a, marine type pitch-blade impellers 100b, helical impellers 100c, and angled pitch-blade type impellers 100d. By agitating the culture medium, such impellers promote the transfer of oxygen, nutrients, and waste products, optimizing the growth and productivity of the biological organisms being cultivated. Shear stress on the organisms imparted by impellers is often a limiting factor in how vigorous mixing can be within a reactor. Improvements are desired in order to have efficient mixing capabilities and to prevent generating deleterious shear for the cells, particularly in large tank applications.

### SUMMARY

Toroidal blades produce low levels of shear stress and increased mass transfer compared to typical impeller/propeller designs. Low shear is advantageous for bioreactor mixing as the microorganisms and cells in the reactor media are shear sensitive. Accordingly, toroidal mixing blades can allow for a higher rate of mixing of the fluid in the reactor to reduce gradients relating to dissolved oxygen, nutrients, pH, and/or temperature to promote uniform growth conditions while increasing mass transfer and still remaining under an operating limit of shear stress. From a bioprocess point of view, creating turbulence is important for generating mixing, but some small-scale eddies (e.g., vortices) create useless shear stress at the cell level without significantly contributing to mixing capabilities. Toroidal impellers create a more uniform and less turbulent flow compared to conventional impellers. Toroidal impellers can also create a more purely axial flow and increased mass transfer in comparison to typical impellers. Although toroidal impellers also reduce cavitation problems, these issues do not occur in cell culture and fermentation. Traditional impellers can generate significant vortex that disrupt mixing and create stagnant zones. Toroidal impellers are designed to minimize these vortices, allowing for more efficient and uniform mixing while generating less shear stress on cells and microorganisms. Toroidal impellers used in mixing applications, for example, in bioreactor applications, distribute energy more effectively throughout the liquid volume, reducing energy losses and increasing mixing efficiency. This means less energy is required to achieve the same level of mixing compared to traditional impellers, resulting in less shear stress on cells and microorganisms. Additionally, toroidal impellers generate fewer radial forces than traditional impellers, reducing additional stress on reactor walls.

The bioreactors and related impellers disclosed herein are suitable for many different potential applications, including brewing, microbiological fermentation (microorganisms, bacteria, yeast), animal cell culture (suspension cells and cells on microcarriers), and plant cell culture, including examples where these cells are genetically modified, infected, transfected, or not. These processes can also include culture mediums comprised of carbon, nitrogen sources, vitamins and minerals, and growth factors; gases such as O2 and CO2; pH control agents; antifoaming agents (or surfactants); and waste products.

Reducing shear stress through the use of the toroidal impellers disclosed herein is not only beneficial for the cells that are sensitive to shear, but also for the products of interest, such as viruses, genetically modified viruses, viral vectors, recombinant proteins, or reagents used in cell culture, such as proteins or transfection mix.

It is also noted that different applications involve differing considerations. For example, in microbial cell applications (e.g., E. coli, S. cerevisiae), oxygen transfer rate (OTR) is the process limiting factor, optimized to hit an OTR that is greater than an oxygen uptake rate (OUR). High rates of mixing create foaming and adverse gradients within the vessel. The use of toroidal impellers in such applications can help balance the need for fast mixing to hit OTR while limiting adverse impact on cells. In another example, in mammalian cell applications (e.g., CHO, HEK293), shearing forces on the cells are of a greater concern. Mammalian cells do not possess a robust cell wall like is present in bacteria and yeast cells. Toroidal impellers can be used in such applications to homogenize the vessel contents while accommodating the shear sensitivity of the cells.

A mixing tank arrangement can include a vessel defining an interior volume; a rotatable shaft assembly extending within the interior volume; one or more toroidal impellers mounted onto the shaft assembly and disposed within the interior volume, the toroidal impeller including a plurality of blade members supported by a central hub extending from a first end to a second axial end, each of the plurality of blade members extending from a first blade end to a second blade end and between an inner side surface and an opposite outer side surface, and being looped such that a first portion of the inner side surface defines a leading face that at least partially faces the first axial end, and such that a second portion of the inner side surface defines a trailing face that at least partially faces the second axial end.

In some examples, at least one of the first and second blade ends of each of the plurality of blade members adjoin an outer surface of the central hub.

In some examples, both the first and second blade ends of each of the plurality of blade members adjoin an outer surface of the central hub.

In some examples, the inner side surface of each of the plurality of blade members defines a radially bounded passageway extending along a second axis disposed at an oblique angle to a longitudinal axis of the toroidal impeller.

In some examples, the plurality of blade members includes at least three blade members.

In some examples, none of the plurality of blade members contacts another of the plurality of blade members.

In some examples, the first end of each of the plurality of blade members is axially separated from the second end of each of the plurality of blade members.

In some examples, the one or more toroidal impellers includes a plurality of toroidal impellers mounted to the shaft assembly.

In some examples, the shaft assembly is supported by a first bearing assembly and a second bearing assembly, and the one or more toroidal impellers is located axially between the first and second bearing assemblies.

A bioreactor can include a vessel defining an interior volume; a motor; a rotatable shaft assembly connected to the motor and extending within the interior volume along a longitudinal axis; one or more impellers including at least one toroidal impeller mounted onto the shaft assembly and disposed within the interior volume, the toroidal impeller including a plurality of blade members supported by a central hub that each define a radially bounded passageway extending along a second axis disposed at an oblique angle to the longitudinal axis.

In some examples, the motor rotates the shaft assembly and the one or more impellers at a maximum RPM of about 1500.

In some examples, the plurality of blade members each include a first end and a second end, wherein at least one of the first and second blade ends of each of the plurality of blade members adjoins an outer surface of the central hub.

In some examples, both of the first and second blade ends of each of the plurality of blade members adjoin an outer surface of the central hub.

In some examples, an inner side surface of each of the plurality of blade members defines the radially bounded passageway.

In some examples, the plurality of blade members includes at least three blade members.

In some examples, none of the plurality of blade members contacts another of the plurality of blade members.

In some examples, the first end of each of the plurality of blade members is axially separated from the second end of each of the plurality of blade members.

In some examples, the at least one toroidal impeller includes a plurality of toroidal impellers mounted to the shaft assembly.

In some examples, the shaft assembly is supported by a first bearing assembly and a second bearing assembly, wherein the at least one toroidal impeller is located axially between the first and second bearing assemblies.

A mixing tank arrangement can include a vessel defining an interior volume; a rotatable shaft assembly extending within the interior volume; and one or more impellers mounted onto the shaft assembly and disposed within the interior volume, the one or more impellers including one or more toroidal impellers. In some examples, the one or more toroidal impellers includes at least one blade member having first and second blade ends, and wherein at least one of the first and second blade ends adjoins an outer surface of a hub. In some examples, both of the first and second blade ends of the at least one blade member adjoin the outer surface of the hub. In some examples, an inner side surface of the at least one blade member defines a radially bounded passageway extending along a second axis disposed at an oblique angle to a longitudinal axis of the one or more toroidal impellers. In some examples, the one or more toroidal impellers includes at least two blade members. In some examples, none of the blade members contacts another of the blade members. In some examples, the first end of at least one blade member is axially separated from the second end of the at least one blade member. In some examples, the one or more impellers includes at least one impeller that is not a toroidal impeller. In some examples, the shaft assembly is supported by a first bearing or bushing assembly and a second bearing or bushing assembly, and the one or more toroidal impellers is located axially between the first and second bearing or bushing assemblies.

A bioreactor can include a vessel defining an interior volume; a motor; a rotatable shaft assembly connected to the motor and extending within the interior volume along a longitudinal axis; one or more impellers mounted onto the shaft assembly and disposed within the interior volume, the one or more impellers including one or more toroidal impellers. In some examples, the motor, during operation, rotates the shaft assembly and the one or more toroidal impellers at a maximum RPM of about 1,500. In some examples, the one or more toroidal impellers includes a plurality of blade members each having a first end and a second end, wherein at least one of the first and second blade ends of each of the plurality of blade members adjoins an outer surface of a hub. In some examples, both of the first and second blade ends of each of the plurality of blade members adjoin the outer surface of the hub. In some examples, an inner side surface of each of the plurality of blade members defines a radially bounded passageway. In some examples, the one or more toroidal impellers includes at least three blade members. In some examples, none of the plurality of blade members contacts another of the plurality of blade members. In some examples, the first end of each of the plurality of blade members is axially separated from the second end of each of the plurality of blade members. In some examples, the one or more impellers includes an impeller that is not a toroidal impeller. In some examples, the shaft assembly is supported by a first bearing or bushing assembly and a second bearing or bushing assembly, and the one or more toroidal impellers is located axially between the first and second bearing or bushing assemblies. In some examples, the one or more impellers includes at least one Rushton type impeller. In some examples, the one or more impellers includes two Rushton type impellers and a single toroidal impeller. In some examples, the single toroidal impeller is located axially between the two Rushton type impellers.

A single use bioreactor can include a flexible bag defining an interior volume; a rotatable shaft assembly extending within the interior volume of the flexible bag; and one or more impellers mounted onto the shaft assembly and disposed within the interior volume, the one or more impellers including one or more toroidal impellers. In some examples, the one or more impellers includes an impeller that is not a toroidal impeller. In some examples, the one or more impellers includes at least one Rushton type impeller.

A bioreactor can include a vessel defining an interior volume; a motor; a rotatable shaft assembly connected to the motor and extending within the interior volume along a longitudinal axis; one or more impellers mounted onto the shaft assembly and disposed within the interior volume, the one or more impellers including one or more toroidal impellers, wherein at least one of the one or more impellers includes surface openings for delivering a gaseous fluid to the interior volume. In some examples, at least some of the surface openings are defined on blades of the one or more impellers. In some examples, at least some of the surface openings are provided on a trailing face of the blades. In some examples, at least some of the surface openings are provided on a leading face of the blades. In some examples, at least some of the surface openings are provided on a central hub portion of the one or more impellers. In some examples, at least some of the surface openings have a size of between 0.15 and 1.0 mm.

A method of fermentation can include providing a bioreactor vessel with one or more toroidal impellers mounted on a rotatable shaft; introducing a fermentation medium into the vessel and inoculating the fermentation medium; rotating the shaft and the one or more toroidal impellers; and introducing a gaseous fluid into the vessel to induce fermentation therein.

In some examples, rotating the shaft is done at a speed between 100 and 1500 RPM.

In some examples, rotating the shaft is done at a speed between 300 and 600 RPM.

In some examples, introducing a gaseous fluid into the vessel is done at a flow rate between 50 L/min and 250 L/min and/or at a flow rate between 0.1 and 1.0 vessel volumes per minute.

In some examples, introducing a gaseous fluid comprises flowing air into the vessel. In some examples, the air is provided at an air flow of 0.5 vvm.

In some examples, inoculating the medium comprises applying one or more bacteria to the medium.

In some examples, the method can include inducing protein expression in the one or more bacteria.

In some examples, the method can include adding fed-batch medium to the vessel at a threshold optical density.

In some examples, the fermentation medium comprises glycerol or glucose as a carbon source.

In some examples, the method is done at a temperature of 28 to 37° C.

In some examples, the method is done at a neutral pH.

A method of enhancing mass transfer in a bioreactor can include providing multiple impellers including at least one toroidal impeller; operating the impellers simultaneously; and achieving volumetric mass transfer coefficient (kLa) values between 60-102 h⁻¹.

In some examples, operating the impellers is done at a speed of up to 500 RPM.

In some examples, the method can include obtaining improved mixing times of 10 to 20 seconds compared to configurations not including a toroidal impeller.

A method of culturing cells in a bioreactor can include providing a liquid with gas bubbles in the bioreactor; agitating the liquid to divide the gas bubbles with at least one toroidal impeller in the bioreactor; delivering the liquid with the divided gas bubbles to a cell culture bed.

A method of low shear mixing a culture medium in a bioreactor can include providing a toroidal impeller in the bioreactor, the toroidal impeller having blade members defining a radially bounded passageway; filling the bioreactor with the culture medium, the culture medium including one or more microorganisms rotating the toroidal impeller to create axial flow with reduced turbulent eddies; mixing the culture medium while maintaining shear stress below levels that damage the microorganisms; and obtaining homogenous distribution of nutrients, gases, and temperature throughout the culture medium.

### BRIEF DESCRIPTION OF THE DRAWINGS

The present invention will be further explained with reference to the appended Figures, wherein like structure is referred to by like numerals throughout the several views.

FIG. 1 is a schematic of a first example of a mixing tank assembly having features in accordance with the present disclosure.

FIG. 2 is a cross-sectional side view of the mixing tank assembly shown in FIG. 1.

FIG. 3 is a schematic of a second example of a mixing tank assembly having features in accordance with the present disclosure.

FIG. 4 is a cross-sectional side view of the mixing tank assembly shown in FIG. 3.

FIG. 5 is a schematic first perspective view of a first example of a toroidal impeller usable with the mixing tanks shown in FIGS. 1 to 4.

FIG. 6 is a schematic second perspective view of the toroidal impeller shown in FIG. 5.

FIG. 7 is a schematic top view of the toroidal impeller shown in FIG. 5.

FIG. 8 is a schematic bottom view of the toroidal impeller shown in FIG. 5.

FIG. 9 is a schematic first side view of the toroidal impeller shown in FIG. 5.

FIG. 10 is a schematic second side view of the toroidal impeller shown in FIG. 5.

FIG. 11 is a schematic cross-sectional side view of the toroidal impeller shown in FIG. 5.

FIG. 12 is a schematic cross-sectional perspective view of the toroidal impeller shown in FIG. 5.

FIG. 13 is a schematic first perspective view of a second example of a toroidal impeller usable with the mixing tanks shown in FIGS. 1 to 4.

FIG. 14 is a schematic second perspective view of the toroidal impeller shown in FIG. 13.

FIG. 15 is a schematic top view of the toroidal impeller shown in FIG. 13.

FIG. 16 is a schematic bottom view of the toroidal impeller shown in FIG. 13.

FIG. 17 is a schematic first side view of the toroidal impeller shown in FIG. 13.

FIG. 18 is a schematic second side view of the toroidal impeller shown in FIG. 13.

FIG. 19 is a schematic cross-sectional side view of the toroidal impeller shown in FIG. 13.

FIG. 20 is a schematic cross-sectional perspective view of the toroidal impeller shown in FIG. 13.

FIG. 21 is a schematic first perspective view of a third example of a toroidal impeller usable with the mixing tanks shown in FIGS. 1 to 4.

FIG. 22 is a schematic second perspective view of the toroidal impeller shown in FIG. 21.

FIG. 23 is a schematic first side view of the toroidal impeller shown in FIG. 21.

FIG. 24 is a schematic second side view of the toroidal impeller shown in FIG. 21.

FIG. 25 is a schematic top view of the toroidal impeller shown in FIG. 21.

FIG. 26 is a schematic bottom view of the toroidal impeller shown in FIG. 21.

FIG. 27 is a third example of a mixing tank assembly having features in accordance with the present disclosure.

FIG. 28 is a schematic perspective view of a prior art Rushton type impeller which may be used in the mixing tank assemblies disclosed herein in combination with the toroidal impellers disclosed herein.

FIG. 29 is a schematic perspective view of a prior art pitch-blade or marine type impeller which may be used in the mixing tank assemblies disclosed herein in combination with the toroidal impellers disclosed herein.

FIG. 30 is a schematic perspective view of a prior art helical type impeller which may be used in the mixing tank assemblies disclosed herein in combination with the toroidal impellers disclosed herein.

FIG. 31 is a schematic perspective view of a prior art angled pitch-blade type impeller which may be used in the mixing tank assemblies disclosed herein in combination with the toroidal impellers disclosed herein.

FIG. 32 is a schematic diagram depicting shear stress in a vessel using a toroidal impeller in accordance with the present disclosure in combination with two Rushton type impellers.

FIG. 33 is a schematic perspective view of a fourth example of a mixing tank assembly having features in accordance with the present disclosure and illustrating an impeller with integrated sparger holes.

FIG. 34 is a schematic perspective view of a fifth example of a mixing tank assembly having features in accordance with the present disclosure and illustrating an impeller with integrated sparger holes.

FIG. 35 is a schematic perspective view of a sixth example of a mixing tank assembly having features in accordance with the present disclosure and illustrating an impeller with integrated sparger holes.

FIG. 36 is a schematic view of a portion of the assembly shown in FIG. 33.

FIG. 37 is a schematic view of a portion of the assembly shown in FIG. 33 with an alternative sparger tube arrangement included.

FIG. 38 is a schematic view of a portion of the assembly shown in FIG. 33 with an alternative sparger tube arrangement included.

FIG. 39 is a schematic view of a portion of the assembly shown in FIG. 33 with an alternative drive shaft arrangement.

FIG. 40 is a schematic view of a portion of the assembly shown in FIG. 33 with an alternative impeller.

FIG. 41 is a chart showing data from the Examples.

FIG. 42 is a chart showing data from the Examples.

FIG. 43 is a chart showing data from the Examples.

FIG. 44 is a chart showing data from the Examples.

FIG. 45 is a chart showing data from the Examples.

FIG. 46 is a chart showing data from the Examples.

FIG. 47 is a chart showing data from the Examples.

FIG. 48 is a chart showing data from the Examples.

FIG. 49 is a chart showing data from the Examples.

FIGS. 50A to 50C are charts showing data from the Examples.

FIGS. 51A to 51B are charts showing data from the Examples.

FIGS. 52A to 52B are charts showing data from the Examples.

FIGS. 53A to 53B are charts showing data from the Examples.

FIGS. 54A to 54B are charts showing data from the Examples.

### DETAILED DESCRIPTION

Herein, example impellers and related mixing assemblies, features, and components therefor are described and depicted. A variety of specific features and components are characterized in detail. Many can be applied to provide advantage. There is no specific requirement that the various individual features and components be applied in an overall assembly with all of the features and characteristics described, however, in order to provide for some benefit in accord with the present disclosure.

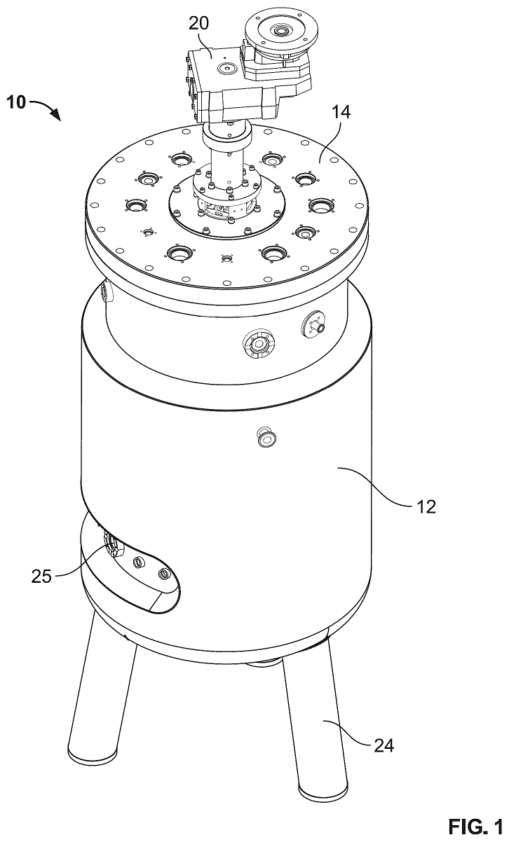

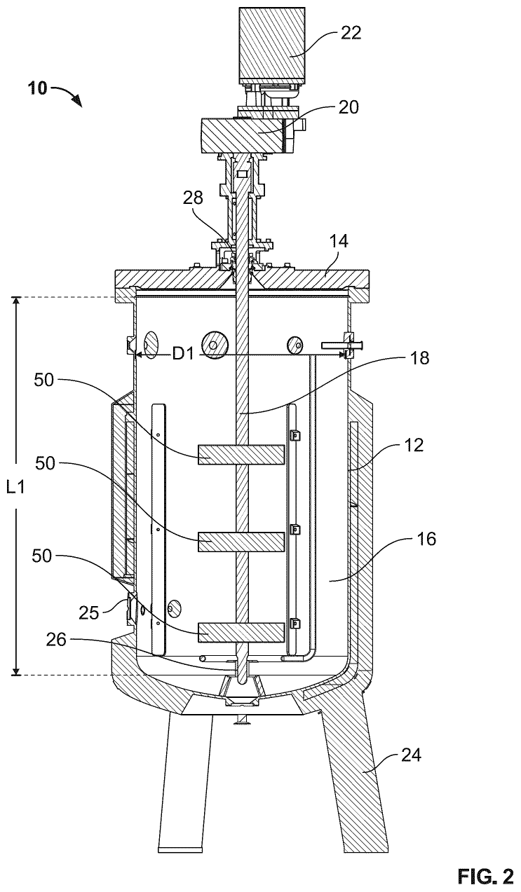

Referring to FIGS. 1 and 2 an example mixing tank assembly 10 is presented. In some examples, the mixing tank assembly 10 can be configured as a bioreactor 10. As shown, the mixing tank assembly 10 includes a vessel 12 and a cover 14 defining an interior volume 16 with various ports 25 for allowing transfer of fluids and other matter. The vessel 12 and cover 14 may be formed from a variety of materials such as polymeric or metal materials (e.g., stainless steel) and may be insulated or non-insulated. In one aspect, the mixing tank assembly 10 includes a vertical shaft 18 driven by a drive assembly 20 mounted to the cover 14. As schematically shown at FIG. 2, the drive assembly 20 can include an electric motor 22. In some examples, the motor 22 can be located at the bottom of the vessel. In one aspect, the shaft 18 is shown as extending through the cover 14 and into the interior volume 16 such that the shaft is supported at each end by bearings or bushings 26, 28. In some examples, the shaft 18 is not a vertical shaft but rather disposed at an oblique angle to a general length of the tank 12 and/or interior volume 16.

To facilitate mixing of a fluid within the interior volume 16, one or more impellers 50 may be mounted to the shaft 18 within the interior volume 16 and between the bearings or bushings 26, 28. In some examples, the shaft 18 extends to or through a center of the impeller 50 while, in other examples, the shaft 18 extends to or through the impeller 50 at a location that is offset from the center of the impeller. In the particular example shown, three vertically spaced impellers 50 are shown. However, more or fewer impellers 50 can be provided depending upon application. For example, two, three, four, five, or six vertically spaced impellers 50 may be provided. Further, the impellers 50 may all be the same or have different types and/or sizes. In one example, the impellers 50 are each configured as impeller 100, described later herein. In other examples, the middle impeller 50 is a toroidal impeller 100 while the upper and lower impellers 50 are prior art type impellers, such as Rushton type impellers 100a, marine type pitch-blade impellers 100b, helical impellers 100c, and angled pitch-blade type impellers 100d. In some examples, a toroidal impeller 100 is located above another type of impeller or multiple impellers. For example, a toroidal impeller 100 can be located above two Rushton type impellers 100a or any other type of impeller (e.g., 100b, 100c, 100d). In some examples, three different types of impellers 50 are provided in which one of the impellers is a toroidal impeller 100. It is further noted that in very large applications, multiple shafts 18 with one or more impellers 50 may be provided within the same interior volume 16 of a vessel 12. It is also noted that the orientation and rotation of the impellers 50 may be selected for desired effect, for example, to provide an upward or downward directed flow within the interior volume.

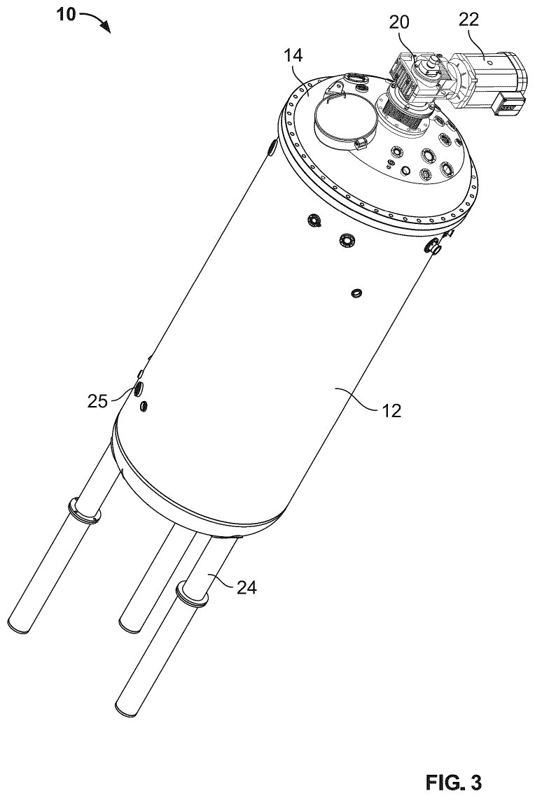

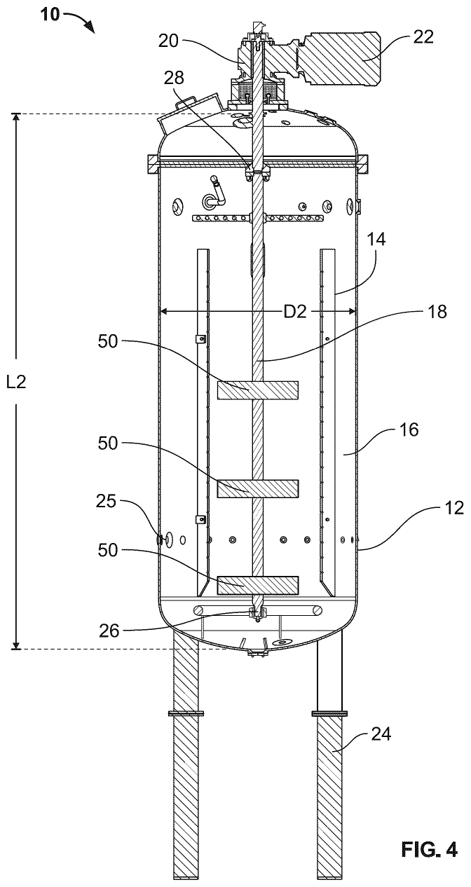

As shown, the vessel 12 and cover 14 define an interior length L1 and an interior diameter D1 in which the Length L1 is greater than the diameter D1 and can therefore be characterized as a vertical or upright vessel. In the example shown, the vessel 12 is supported by a plurality of legs 24. In the particular example shown, the vessel has an interior volume of about 400 liters. Referring to FIGS. 3 and 4, a second example of a mixing tank assembly 10 is shown with the same general construction as that shown in FIGS. 1 and 2. However, the vessel 10 in FIGS. 3 and 4 has a length L2 and diameter D2 that define significantly greater volume of about 4000 liters, primarily due to a greater length L2.

Referring to FIGS. 5 to 10, a first example of a toroidal impeller 100 extending along a longitudinal axis X is presented. In one aspect, the toroidal impeller 100 includes a central hub portion 102 defining a central opening 102c and extending between first and second axial ends 102a, 102b. The central opening 102c is configured to receive the shaft 18. The central hub portion 102 may be fixed to the shaft 18 by any means, such as by welding or mechanical fasteners. As shown, the longitudinal axis X of the toroidal impeller is coaxial with the length of the shaft 18 and rotates about the longitudinal axis X in a rotational direction R. As noted above, the impellers 100 can be configured to rotate in the opposite direction of direction R for desired effect. In some applications, the shaft 18 and impellers 100 rotate at speeds between 100 and 1000 revolutions per minute (RPM). In some applications, the outermost diameter of the toroidal impeller 100 is within about 10 to 90 percent of the internal diameter D1, for example within about 20 percent to 30 percent.

In one aspect, the toroidal impeller 100 includes a plurality radially extending blade members 110 supported by the central hub portion 102. In the example shown, three blade members 110 are provided. Other numbers of blade members are possible. For example, the toroidal impeller 100 could be provided with two blade members, four blade members, five blade members, six blade members, or more than six blade members. As shown, each of the blade members 110 extends from a first end 110a to a second end 110b. Each blade member 110 also defines a first side surface 110d, an opposite second side surface 110e, a first side edge 110f, and an opposite second side edge 110g, each of which extends between the first and second ends 110a, 110b. In some characterizations, the first side surface 110d can be referred to as an inner side surface 110d while the second side surface 110e can be referred to as an outer side surface 110e.

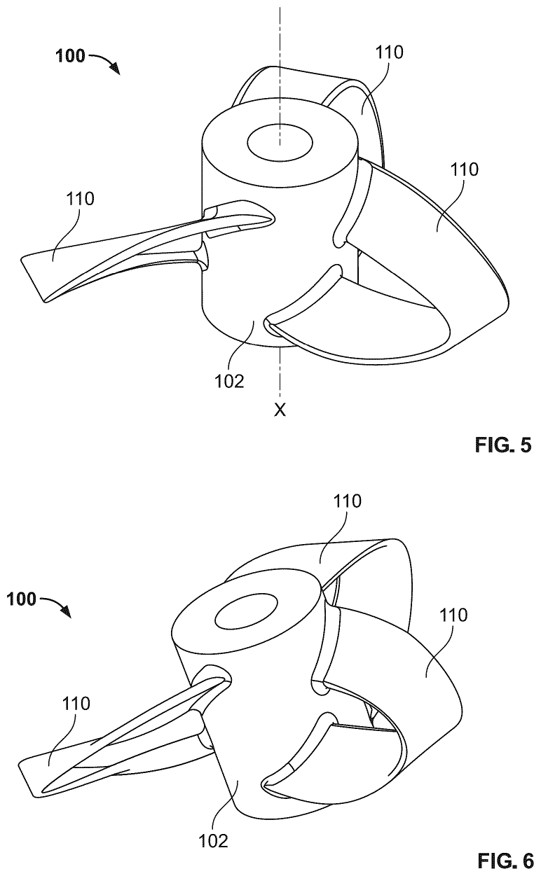

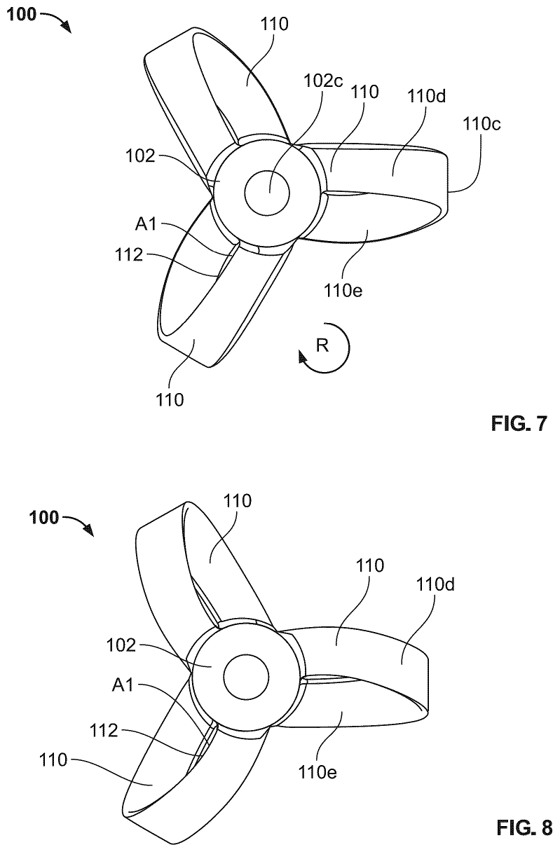

As shown, each of the blade members 110 extends in a looping or folding fashion such that each end 110a, 110b adjoins an outer surface 102d of the central hub portion 102. In one aspect, each blade member 110 extends to a radially distal portion 110c that is located axially between the first and second ends 110a, 110b. The radially distal portions 110c of the blade members 110 also define the outermost radial points of the toroidal impeller 100 as a whole. With such a configuration, each blade member 110 defines a radially bounded passageway 112. By use the term ‘radial bounded passageway’ it is meant to define a passageway that is closed (i.e., not open) at a radially outward edge of the passageway. As can be seen at FIGS. 7 and 8, the radially bounded passageway 112 has a line-of-sight open area A1 in the axial direction and a line-of-sight open area A2 in the radial direction, wherein the area A2 is greater than the area A1. In one aspect, the radial bounded passageway 112, in general, extends along an average axis X2 that is at an oblique angle to the longitudinal axis X and extends towards the first end 102a of the central hub in a direction from the side edge 110f towards side edge 110g. Stated another way, the radial bounded passageway 112 extends upwardly towards the first end 102a in a direction from side edge 110f towards side edge 110g. Due to such a configuration, toroidal impellers of the type described herein can induce substantial axial flow within the vessel for optimized mixing.

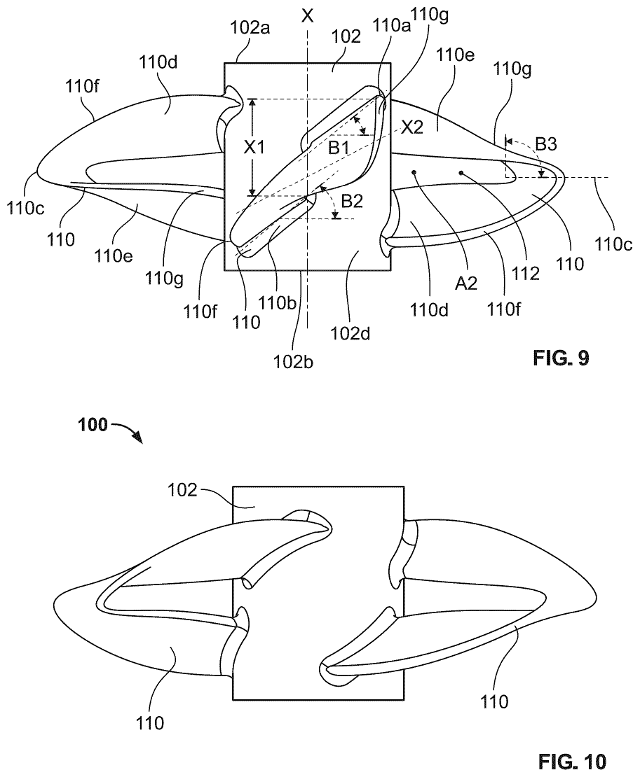

In the example shown, the first and second ends 110a, 110b are at least partially axially separated such that corresponding points on the ends 110a, 110b adjoin the outer surface 102d at separate axial locations. For example, and as illustrated at FIG. 9, the side edge 110g at the first end 110a of the blade member 110 adjoins outer surface 102d at a separate axial location by a distance X1 in comparison to the side edge 110g at the second end 110b of the same blade member 110. With continued reference to FIG. 9, it can also be seen that the first end 110a adjoins the outer surface 102d at a first angle B1 relative to a line parallel to the longitudinal axis X while the second end 110b adjoins the outer surface 102d at a second angle B2 relative to a line parallel to the longitudinal axis X. In the example shown, angles B1 and B2 are substantially equal, meaning that they are at least within 5 degrees of each other. In one example, angles B1 and B2 are about 50 degrees. It can also be seen in FIG. 9 that a line passing through the edges 110f, 110g at the distal end 110c of each blade member 110 is disposed at an angle B3 relative to a line parallel to the longitudinal axis X. In the example shown, angle B3 is about 90 degrees. As most easily seen at FIGS. 5 and 6, each blade member 110 twists or curves from the angle B1 at the first end 110a, folds through the distal end 110c at angle B3, and twists or curves to the angle B2 at the second end 110b to form a toroidal shape. In some examples, the blade members 110 can have a generally constant thickness or can have a varying thickness, such as an airfoil type shape. In some examples, one or both of the surfaces 110d, 110e can be provided with a camber defining a curved cross-sectional profile between edges 110f, 110g at a particular radial location, or can be configured to have a generally planar surface defining a straight cross-sectional profile between edges 110f, 110g at a particular location.

Due to the folding shape of the blade members 110, and as most easily seen at FIG. 9, portions of each side surface 110d, 110e form a leading and trailing face during rotation of the toroidal impeller 100. Stated another way, and as most easily viewed at FIGS. 7 and 8, a portion of each side surface 110d, 110e faces generally towards first axial end 102a while another portion of each side surface 110d, 110e faces generally towards the second axial end 102b. By use of the term ‘faces generally towards’ with respect to the surfaces 110d, 110e, it is meant that the specified surface is at least viewable from an axial top or bottom end of the impeller in a direction orthogonal to the longitudinal axis. In one characterization, a leading face can be defined as one that at least partially faces in the direction of rotation R while a trailing face can be defined as one that at least partially faces in a direction opposite the direction of rotation R. In another aspect, the blade members 110 can be described as having a side surface 110d that is always an interior-facing side of the blade member 110 and having an opposite side surface 110e that is always an exterior-facing side surface.

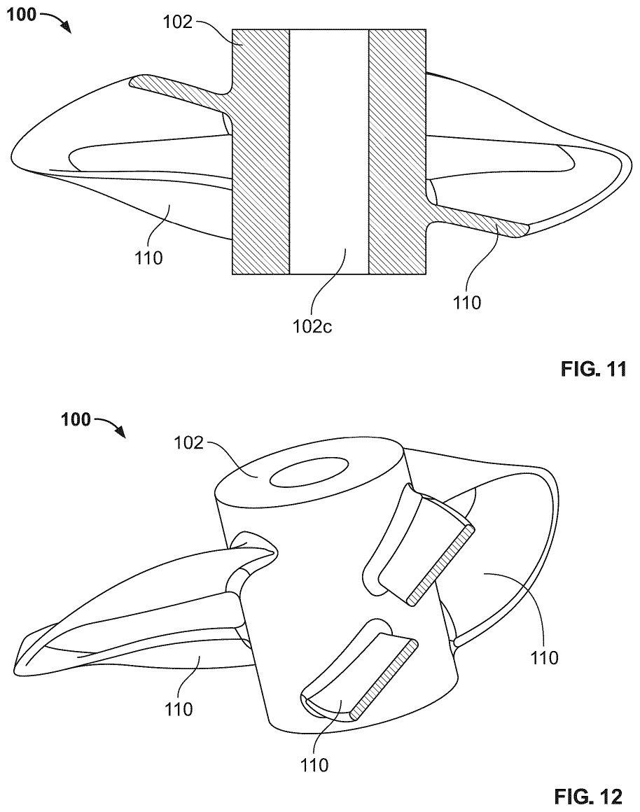

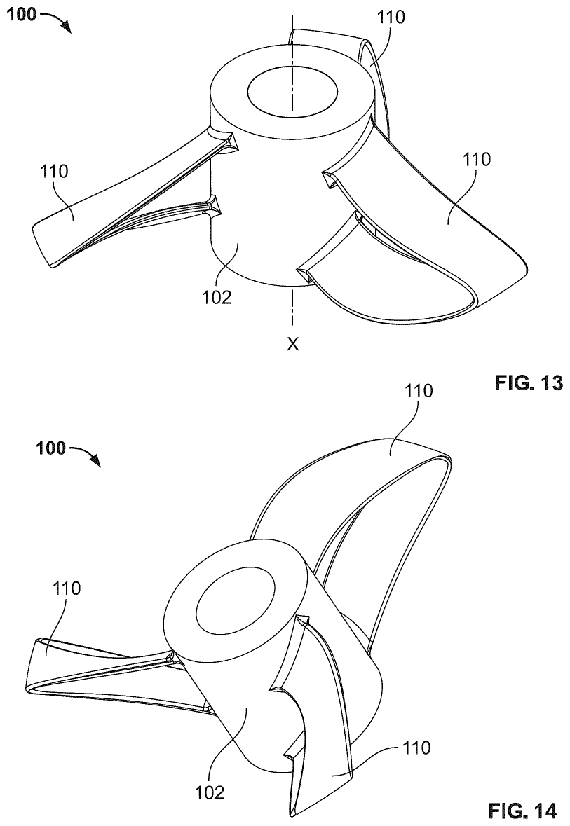

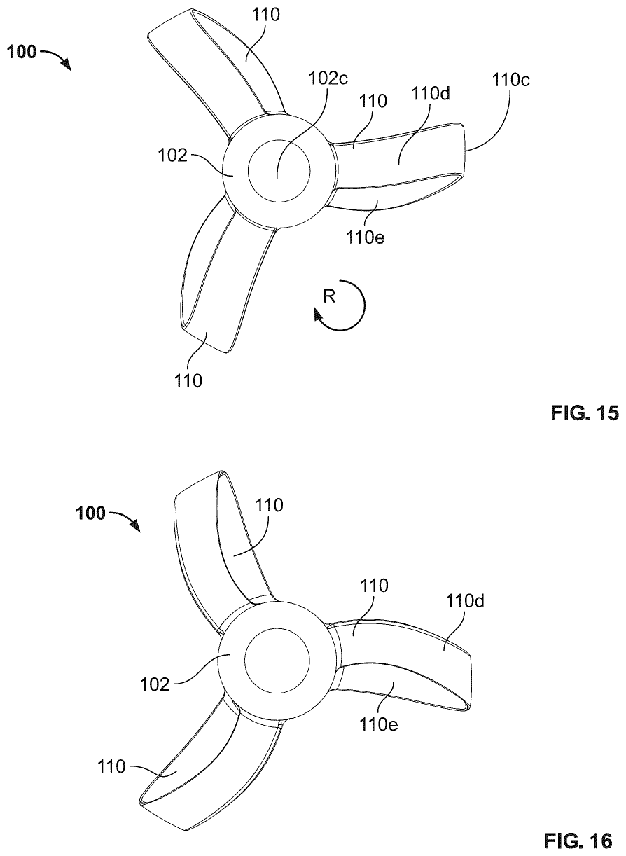

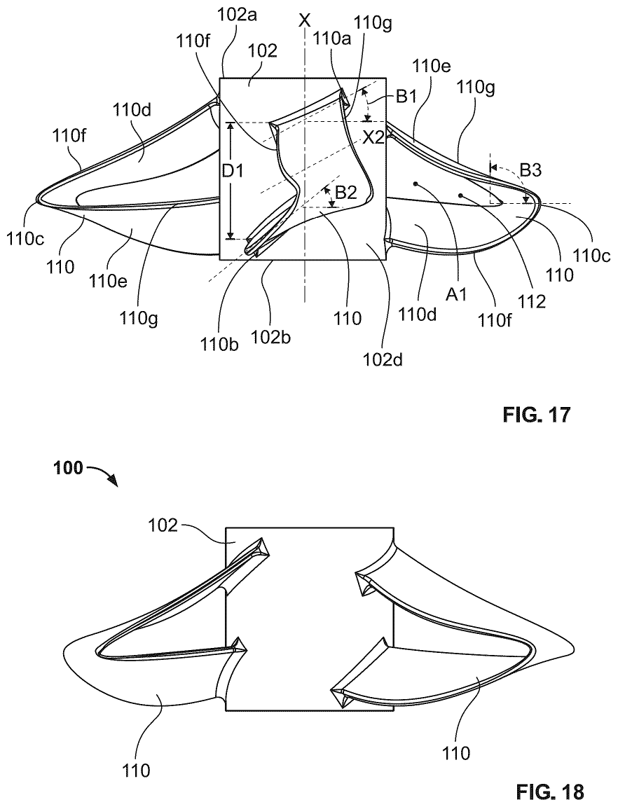

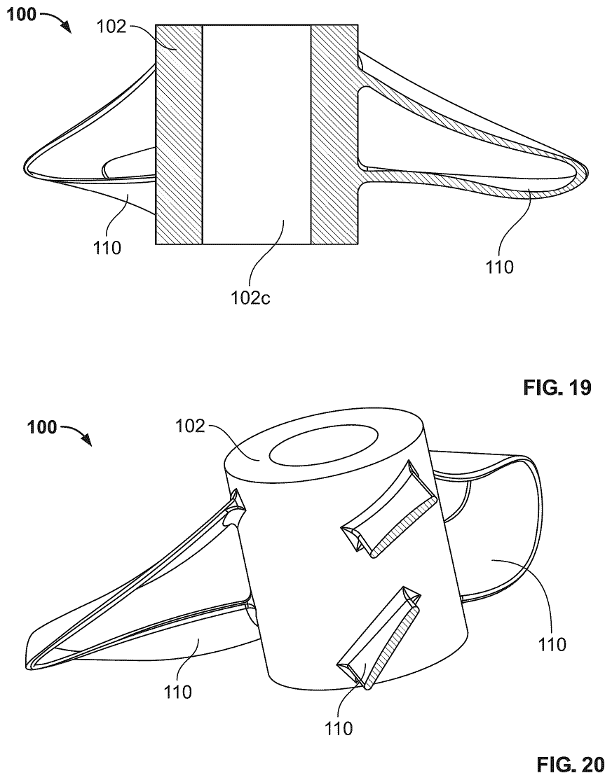

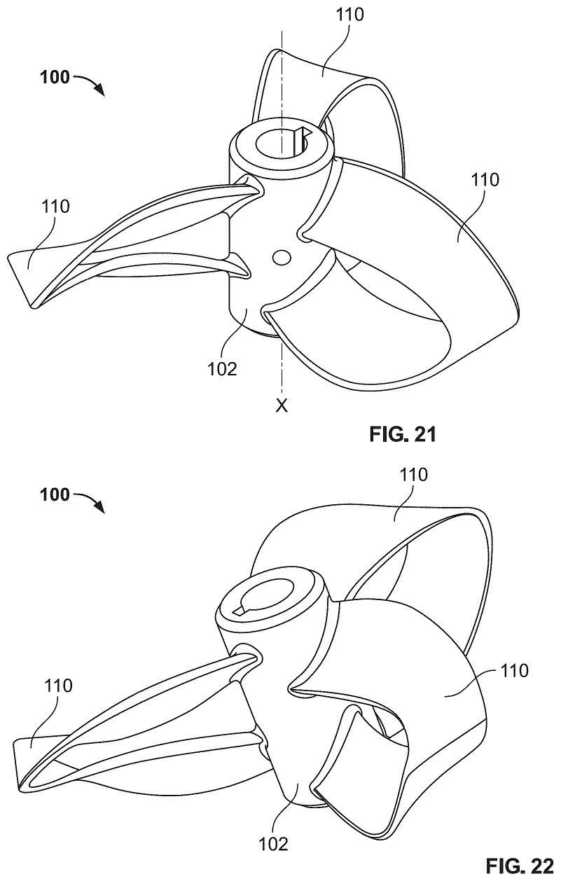

With reference to FIGS. 13 to 20, a second example of a toroidal impeller 100 is presented. Many similarities exist between the impeller 100 of FIGS. 5 to 12 and the impeller 100 of FIGS. 13 to 20. Accordingly, the above-provided description is fully applicable for the impeller 100 of FIGS. 13 to 20 except for the differences explained herein. As detailed at FIG. 17, one such difference is that the angles B1 and B2 have a larger difference from each other, and in the example shown a difference of about 10 to 15 degrees with B2 being about 50 degrees and B1 being about 65 degrees. Accordingly, the first ends 110a of the blade members 110 are disposed at a greater angle to the longitudinal axis X in comparison to the second ends 110b for the embodiment shown in FIGS. 5 to 12. Also, as can be seen in FIGS. 15 and 16, the curvature of the blade members 110 is shaped such that the radial bounded passageway 112 has no associated line-of-sight opening area A1 in the axial direction, while FIG. 17 shows a slightly larger line-of-sight opening area A1 in the radial direction in comparison to the embodiment shown in FIGS. 5 to 12. FIG. 17 also shows that, in comparison to the previously described embodiment, the location the line defining angle B3 at the radial distant portion is located axially closer to the second end 110b rather than being roughly equidistant between ends 110a, 110b in the first example.

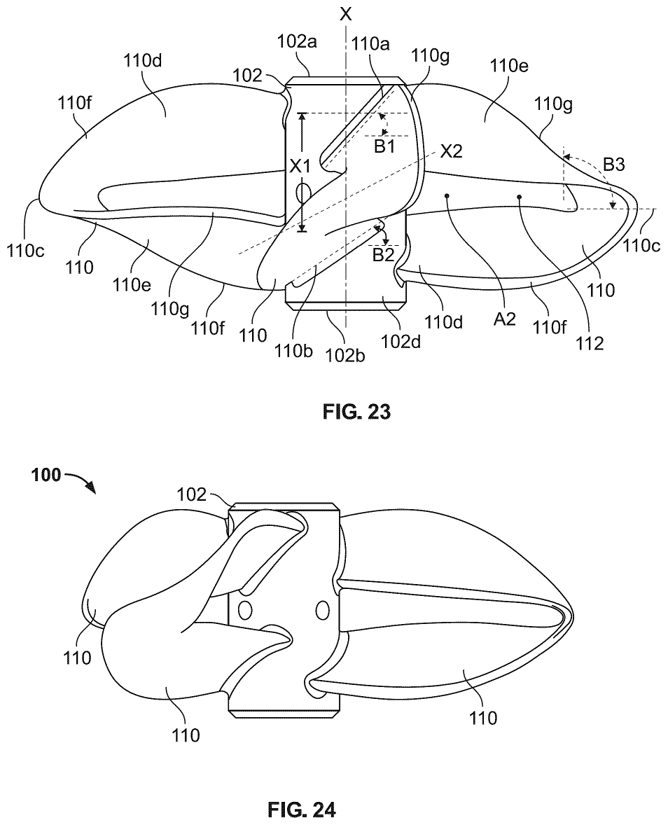

With reference to FIGS. 21 to 26, a third example of a toroidal impeller 100 is presented. Many similarities exist between the impellers 100 of FIGS. 5 to 22 and the impeller 100 of FIGS. 21 to 26. Accordingly, the above-provided descriptions are fully applicable for the impeller 100 of FIGS. 21 to 26 except for the differences explained herein. As detailed at FIG. 23, one such difference is that the angles B1 and B2 are presented at different angles, wherein angle B1 is about 50 degrees and B2 being about 27.5 degrees, resulting in a difference of about 23.5 degrees. Accordingly, the first ends 110a of the blade members 110 are disposed at a greater angle to the longitudinal axis X in comparison to the second ends 110b for the embodiment shown in FIGS. 5 to 12. Also, similarly to the embodiment shown at FIGS. 13 to 20 and in contrast to the embodiment shown at FIGS. 5 to 22, each blade 110 at least axially overlaps itself at the ends 110a and 110b. By using the term “axially overlaps,” it is meant that an axial line parallel to the longitudinal axis X can pass through at least a portion of the blade 110 at end 110a and through at least a portion of the same blade 110 at end 110b. Stated another way, the edge 110f of each blade 110 proximate the end 110a is located radially between the edges 110f, 110g of the same blade 110 proximate end 110b.

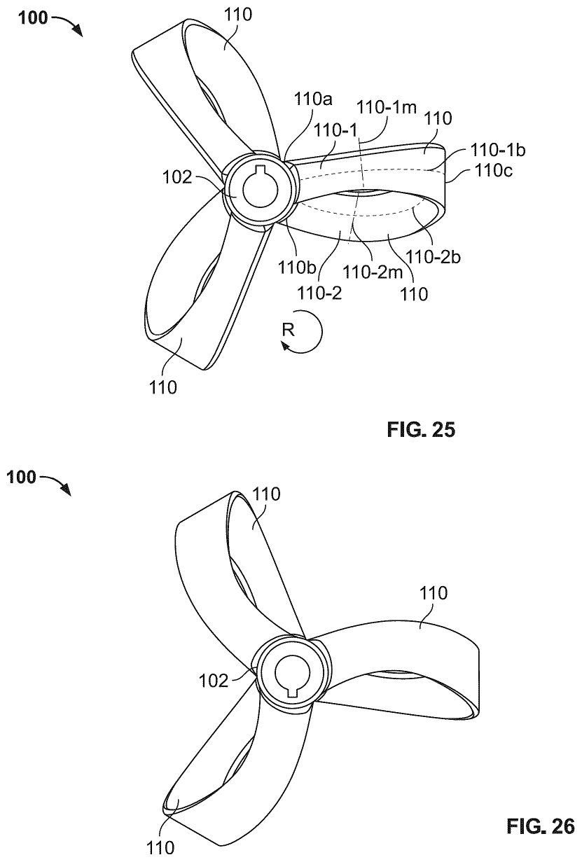

With reference to FIG. 25, each blade 100 can be characterized as forming a first vane 110-1 extending between the first end 110a and distal end 110c and thus having a pitch angle that varies between angles B1 and B3. Each blade 110 can also be characterized as forming a second vane 110-2 extending between the second end 110b and distal end 110c and thus having a pitch angle that varies between angles B2 and B3. In the example shown, the pitch angle of the first vane 110-1 gradually increases from angle B1 to a maximum angle B4 at a midpoint location 110-1m between ends 110a, 110c and then gradually decreases to the angle B3 as the first vane 110-1 extends to the end 110c. Similarly, the pitch angle of the second vane 110-2 gradually increases from angle B2 to a maximum pitch angle at a midpoint location 110-2m between ends 110b, 110c and then gradually decreases to the angle B3 as the second vane 110-2 extends to the end 110c. In some examples, the midpoint location 110-1m is located closer to end 110a than to end 110c. In some examples, the midpoint location 110-1m is located closer to end 110c than to end 110a. In some examples, the midpoint location 110-1m is located within the middle third of the distance between ends 110a and 110c. In some examples, the midpoint location 110-2m is located closer to end 110b than to end 110c. In some examples, the midpoint location 110-2m is located closer to end 110c than to end 110b. In some examples, the midpoint location 110-2m is located within the middle third of the distance between ends 110b and 110c. In some examples, B1 is the maximum pitch angle for the first vane 110-1. In some examples, B2 is the maximum pitch angle for the second vane 110-2. It is noted that the above-described pitch angles and locations for each of the vanes 110-1 and 110-2 may be provided in any combination without departing from the concepts presented herein.

The design of the toroidal impeller 110 can be optimized for different operating conditions based on the characteristics of the culture medium. For example, a higher oxygen transfer rate can be achieved by increasing the angle B1. In examples, angles of greater than 40 degrees have been found to facilitate high rates of oxygen transfer. High rates of mixing and fluid mass transfer can be achieved with a relatively more shallow pitch relating to angle B2 although decreasing angle B2 does have a decreasing effect on the OTR (oxygen transfer rate). In some examples, a shallow pitch angle B2 of 35 degrees is used in order to more gradually increase the velocity of the fluid being moved by the impeller, before it is more radically moved by the second vane. It has been further observed that providing the blade pitch B1 of 40 degrees provides for a significant improvement to OTR in comparison to a blade pitch B2 of around 30 degrees.

In another aspect, and with continued reference to FIG. 25, it can be viewed that the second vane 110-2 has a more rounded path, as defined by a longitudinal bisecting line 110-2b. This provides effective fluid mass transfer at lower rates of stress placed on the fluid. In comparison, the first vane 110-1 has a more straight path, as defined by a longitudinal bisecting line 110-1b, which provides the more blunt blade effects that facilitate high OTR.

In view of the above, the term ‘toroidal impeller’ may be characterized with one or more of the following definitions: an impeller having at least one blade member that defines a radially bounded passageway; an impeller having at least one blade member extending between first and second ends, wherein the blade member is folded such that first end extends to a hub member and the second end extends to the hub member or to an adjacent blade member proximate the hub member; an impeller having at least one blade member with opposite first and second side surfaces, wherein the blade is folded such that the first side surface is always inwardly facing and the second side surface is always outwardly facing; and/or as an impeller having at least one blade member with opposite first and second side surfaces wherein the blade is folded such that each of the first and second side surfaces presents a leading and trailing face of the blade member. The toroidal impeller may also be referred to as an agitator or toroidal agitator.

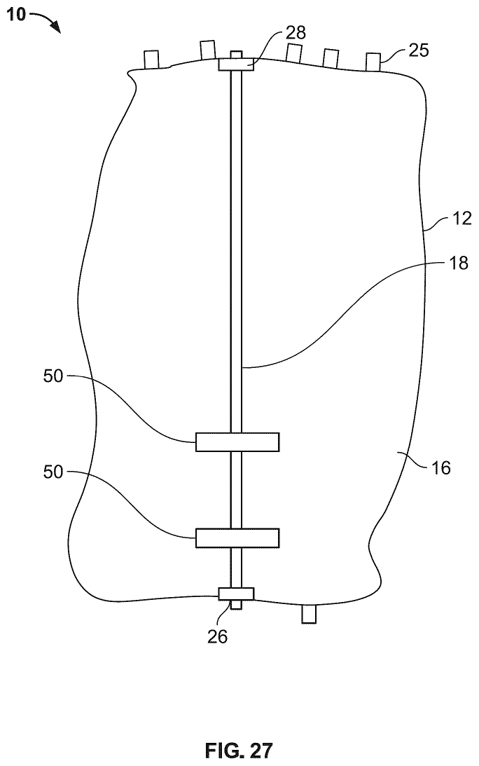

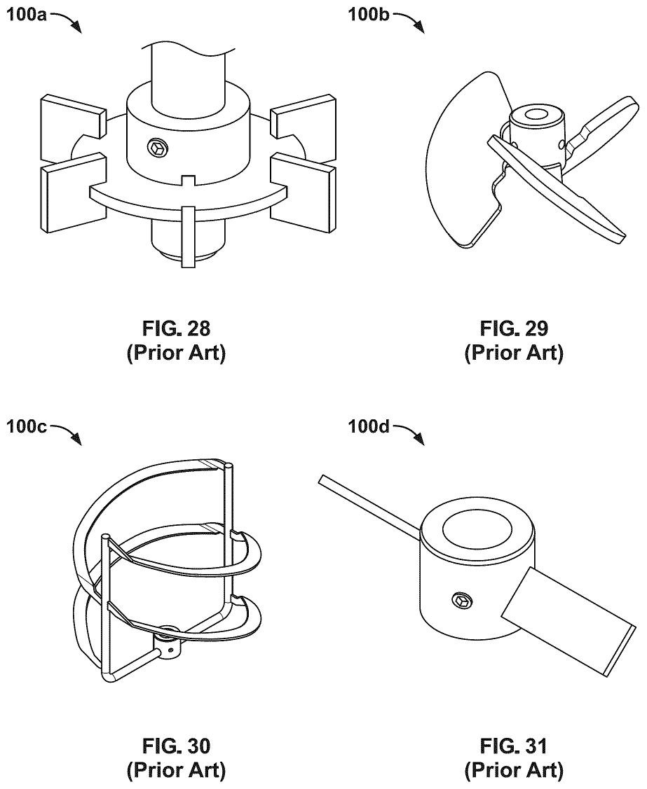

Referring to FIG. 27, a further example of a mixing tank 10 is presented in which the vessel 12, shaft 18, bushings or bearings 26, 28, and impellers 50 are provided as a single use, disposable assembly that can be received in a machine having the electric motor 22 and related controls. With such a configuration, the vessel 12 is provided as a flexible bag or bladder 12. In some examples, the flexible bag or bladder 12 is formed from a polymeric material, such as a plastic and/or rubber type material. In some examples, the flexible bag or bladder 12 has an interior volume 16 of between 50 liters and 2,000 liters. As with other described examples, the mixing tank 10 can be provided with a variety of different impeller combinations and in the example shown is provided with two impellers 50. More or fewer impellers 50 may be provided, such as one impeller or three, four, or five impellers. In some examples, all of the impellers 50 are toroidal impellers 100. In some examples, the impellers 50 include one or more toroidal impellers 100 in combination with one or more Rushton type impellers 100a, marine type pitch-blade impellers 100b, helical impellers 100c, and angled pitch-blade type impellers 100d, as illustrated at FIGS. 28 to 31. FIG. 27 also shows that the flexible bag or bladder 12 may be provided with various ports 25 for transferring fluids (liquid and/or gas) or other matter to and from the interior volume 16.

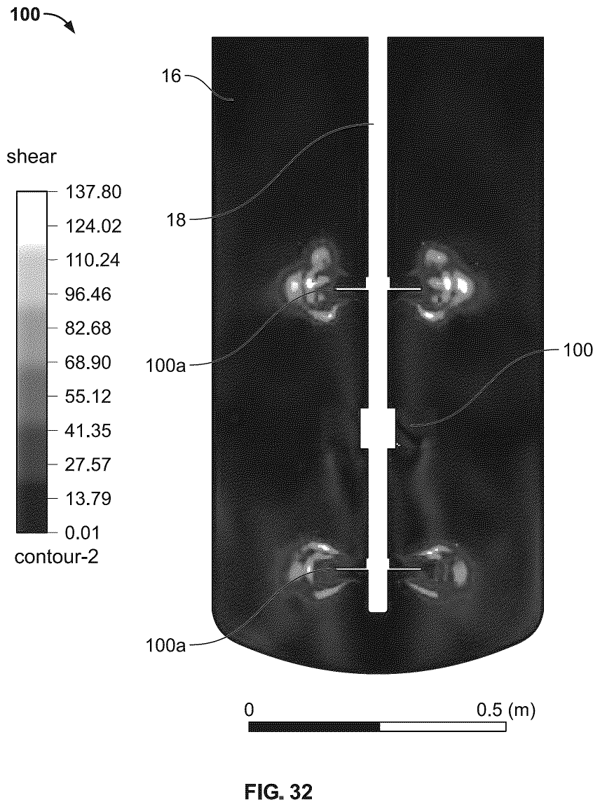

Referring to FIG. 32, a schematic is presented showing a computational fluid dynamic model during a mixing operation using three vertically spaced impellers in which the upper and lower impellers are Rushton type impellers 100a and the central impeller is a toroidal impeller 100 of the present disclosure. As can be seen in FIG. 32, the shear attributable to the toroidal impeller 100 is far lower than that associated with the Rushton type impellers 100a. Further, in comparison to a typical prior art configuration, calculations have shown that while localized peak shear stress can be higher with the configuration shown in FIG. 32, the overall average shear stress is significantly lower, thereby resulting in an improved mixing operation. Modelling also shows that initial mixing time using at least one toroidal impeller can be improved by about ten percent in comparison to configurations using prior art type impellers, such as marine type impellers.

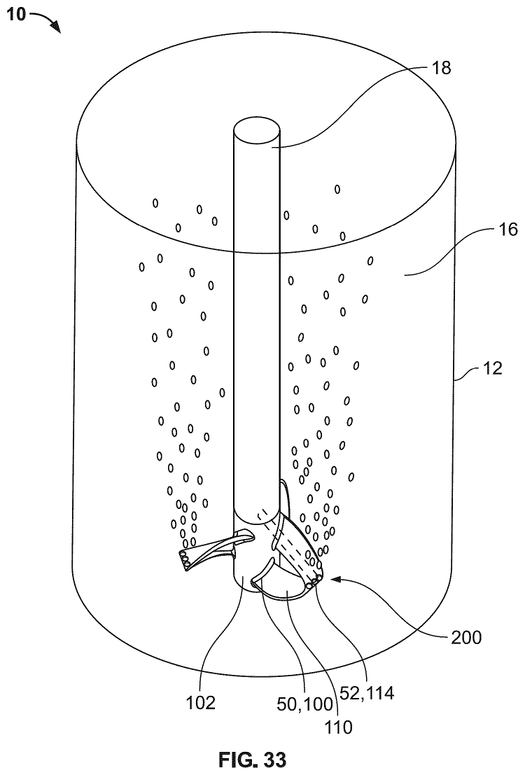

With reference to FIGS. 33 to 40, and as described further in this section, the above described mixing tank assemblies 10 can be provided with a sparger arrangement 200 that is either incorporated into the impeller 50 or provided as a separate tube within the tank interior volume 16. A sparger in a bioreactor tank system is a device used to introduce gases, typically air or oxygen, into the liquid medium within the tank. The main function of a sparger is to provide efficient aeration and mixing of the liquid medium, which is essential for the growth and metabolism of microorganisms in bioprocesses. In examples, the sparger arrangement 200 includes a series of small holes or nozzles. The gas, such as air or oxygen, is introduced through the sparger arrangement 200, and as it passes through the pores or holes, it forms small bubbles in the liquid medium. These bubbles rise to the surface, creating a gentle agitation and mixing of the medium. The sparger serves several important functions in a bioreactor tank system. For example, the introduction of air or oxygen through the sparger provides the necessary oxygen supply for the aerobic microorganisms in the bioreactor. Oxygen is a vital component required for cellular respiration and energy production. Additionally, the rising bubbles generated by the sparger create a circulation and mixing effect in the liquid medium. This helps in distributing nutrients evenly, maintaining a homogeneous environment, and preventing the formation of concentration gradients within the tank. The small bubbles formed by the sparger arrangement 200 also increase the liquid-gas interface area, facilitating efficient mass transfer of gases into the liquid medium. This enhances the exchange of gases, such as oxygen and carbon dioxide, between the air and the culture medium, promoting metabolic activities of the microorganisms. The sparger arrangement and operation can also be adjusted to control the level of shear stress on the microorganisms. By regulating the gas flow rate through the sparger arrangement 200, the shear stress can be optimized to avoid cell damage while ensuring sufficient mixing and aeration. Overall, the sparger arrangement 200 plays a critical role in maintaining optimal growth conditions for microorganisms in a bioreactor tank system, ensuring efficient aeration, mixing, and mass transfer, which are essential for successful bioprocesses. By combining the sparger arrangement 200 into the impeller 100, as shown and described herein, significantly improvements in oxygen transfer can be obtained while keeping shear stresses minimized. For example, and in one aspect, the positioning of openings associated with the sparger arrangement 200 on or near the blades of the impeller 100 exposes the gaseous bubbles to turbulence and shearing action created by the rotating blades which advantageously serves to divide the bubbles into more numerous bubbles having a smaller size.

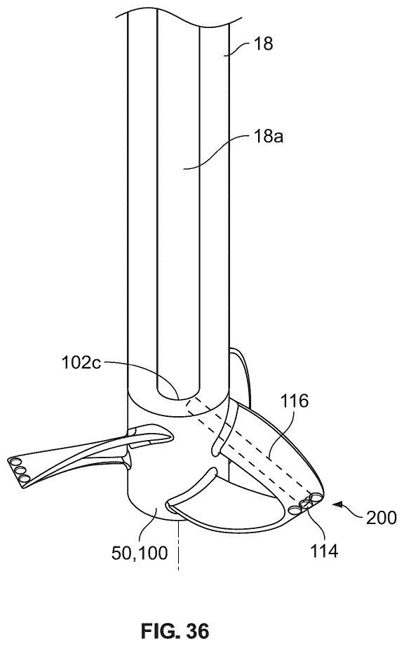

As presented at FIG. 33, the sparger arrangement 200 is integrated into the impeller 50, 100 such that a plurality of holes or openings 52, 114 are provided on the surface of one or more of the blades 110. With such an arrangement, and as further illustrated schematically at FIG. 36, each of the blades 110 is provided with openings 114 in fluid communication with an internal passageway 116 that is in fluid communication with a passageway defined within the central hub portion 102, for example the central opening 102c. The passageway defined in the hub portion 102 is in fluid communication with a passageway 18a defined in the shaft 18 such that air, oxygen, or another gaseous fluid can be pumped into the interior volume 16 of the tank 12 via the shaft 16 and impeller 100. In some examples, the gaseous fluid is pumped into the shaft at a pressure of between 1 and 6 bar, and in some cases at a pressure between 1 and 2 bar. In some examples, the shaft 18 may be coupled to the electric motor 22 via a magnetic coupling to more easily facilitate transporting and delivering a gaseous fluid through the hollow shaft 18.

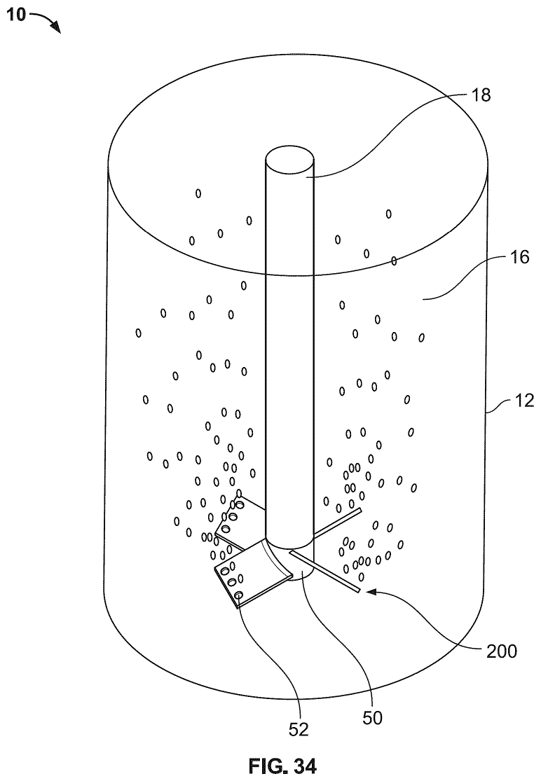

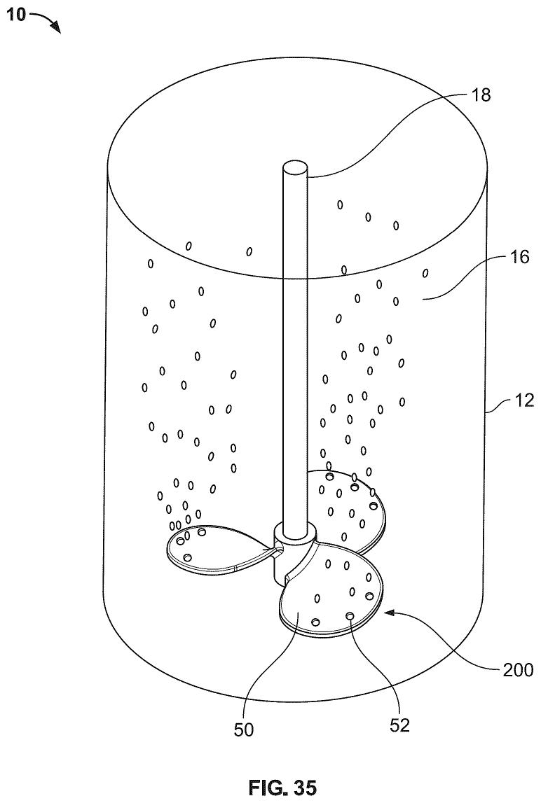

The above-described concept is not limited to toroidal impellers and may be applied to other types of impellers in generally the same manner. For example, FIG. 34 shows a pitched blade impeller 50 while FIG. 35 shows a marine type impeller 50 provided with openings 52 in the blades.

With reference to FIG. 36, it can be seen that the openings 114 are provided on the surface 110e of each blade 110 at a location that is between the end 110a and the radially distal portion 110c and that is proximate the radially distal portion 110c. Many locations for the openings 114 are possible. For example, the openings 114 could be located only on the surface 110e, only on the surface 110d, or on both of surfaces 110e, 110d. Further, openings 114 could be located across the entirety or only a portion of surfaces 110e and/or 110d. In some examples, the openings are located on a trailing face or a leading face of each blade 110. In some cases, the openings 114 can be on an upwards facing surface. In some cases, the openings 114 can be on a downwards facing surface. In some cases, the openings 114 can be on a trailing face. In some cases, the openings 114 can be on a leading face.

In some examples, the openings 114 are all the same size and/or shape. In some examples, at least some of the openings are differently sized and/or shaped from others of the openings. In some examples, the openings 114 have a size that is between 0.15 millimeters (mm) and 1.0 mm. In some examples, the impeller 50, 100 is formed by investment casting. In some examples, the impeller 50, 100 is formed by additive manufacturing. In some examples, the openings 52, 114 are formed in the impeller 50, 100 by a machining process. In some examples, all or a portion of the blades 110 are porously formed, for example, by an additive manufacturing process to define the openings 114. In some examples, the central hub portion 102 is additionally or alternatively porously formed.

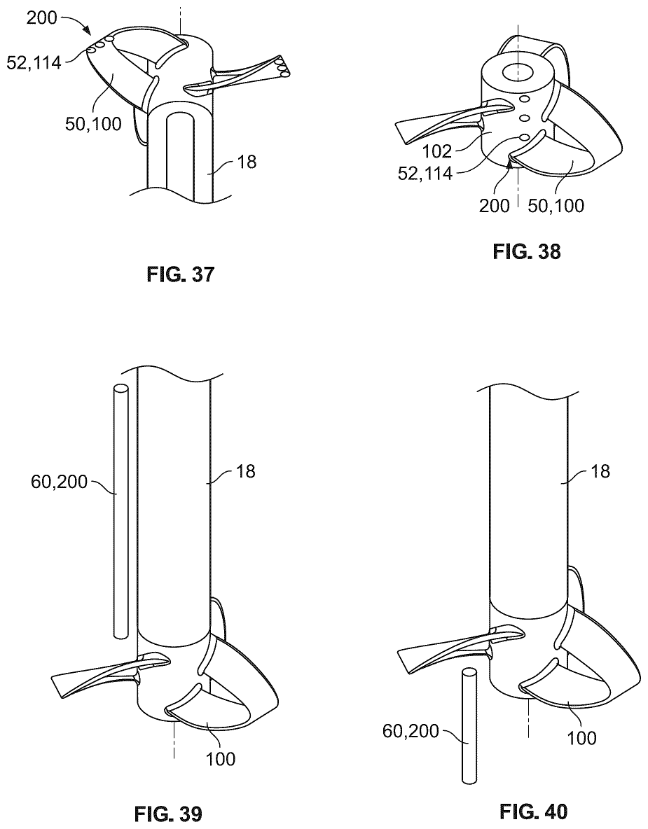

Referring to FIGS. 37 to 40, alternative sparger arrangements 200 are presented. FIG. 37 illustrates that the shaft 18 may extend to the impeller 50, 100 from the bottom side of the impeller, while FIG. 38 illustrates that the openings 52, 114 may be provided alternatively or additionally on the hub portion 102 of the impeller. FIGS. 39 and 40 illustrate that the sparger arrangement 200 may be provided as a separate tube 60 in combination with a toroidal impeller 100. FIG. 39 illustrates the end of the tube 60 extending to and discharging a gas towards a top side of the impeller 100, while FIG. 40 illustrates the tube 60 extending to and discharging a gas towards a bottom side of the impeller 100.

In some examples, a mixing tank assembly 10 is provided with one or more impellers 50, 100, wherein at least one of the impellers 50, 100 is provided with openings 52, 114. In some examples, the impeller 50, 100 having the openings 114 is the bottom-most impeller 114 in the tank 12.

### EXAMPLES

Various embodiments of the present disclosure can be better understood by reference to the following Examples which are offered by way of illustration. The present disclosure is not limited to the Examples given herein.

Example bioreactors were assembled and tested using the toroidal impellers discussed herein. Comparative bioreactors were also assembled and tested as described in the Examples below. The testing Examples below include both 10 L batches and 500 L batches tested with several different impellers, including the toroidal impellers discussed above.

### Example 1. Toroidal Impeller Comparison

In Example 1, an initial comparison of various types of impellers was run in a bioreactor. Here, the same size bioreactor compartment was run with the same load, except for several different types of impellers so as to compare the toroidal impeller (such as shown in FIG. 5) to conventional pitch blade (such as illustrated in FIG. 29) and Rushton type (such as illustrated in FIG. 28) impellers.

The various batches were run with water or water with 0.5% by weight xanthan gum, at agitation of 100 RPM, 150 RPM or 200 RPM, and with airflow ranging from 50 L/min to 250 L/min or, stated another way 0.1 to 0.5 vessel volumes per minute (VVM). In examples, the airflow rate can range between 0.1 to 1.0 VVM. The batches that were run for Example 1 are summarized in the below Table:

TABLE 1
Example 1 samples

| | | | | Air | |
|---|---|---|---|---|---|
| # | Impeller | Liquid | Agitation | Flow | |
| P1 | Pitch Blade | Water | 150 RPM | 50 | L/min |
| P2 | | | | 125 | L/min |
| P3 | | | | 250 | L/min |
| P4 | | | 200 RPM | 50 | L/min |
| P5 | | | | 125 | L/min |
| P6 | | | | 250 | L/min |
| P7 | | | 250 RPM | 50 | L/min |
| P8 | | | | 125 | L/min |
| P9 | | | | 250 | L/min |
| P10 | | Xanthan | 150 RPM | 50 | L/min |
| P11 | | Gum | | 125 | L/min |
| P12 | | | | 250 | L/min |
| P13 | | | 200 RPM | 50 | L/min |
| P14 | | | | 125 | L/min |
| P15 | | | | 250 | L/min |
| P16 | | | 250 RPM | 50 | L/min |
| P17 | | | | 125 | L/min |
| P18 | | | | 250 | L/min |
| R1 | Rushton | Water | 100 RPM | 50 | L/min |
| R2 | | | | 125 | L/min |
| R3 | | | | 250 | L/min |
| R4 | | | 150 RPM | 50 | L/min |
| R5 | | | | 125 | L/min |
| R6 | | | | 250 | L/min |
| R7 | | | 200 RPM | 50 | L/min |
| R8 | | | | 125 | L/min |
| R9 | | | | 250 | L/min |
| R10 | | Xanthan | 100 RPM | 50 | L/min |
| R11 | | Gum | | 125 | L/min |
| R12 | | | | 250 | L/min |
| R13 | | | 150 RPM | 50 | L/min |
| R14 | | | | 125 | L/min |
| R15 | | | | 250 | L/min |
| R16 | | | 200 RPM | 50 | L/min |
| R17 | | | | 125 | L/min |
| R18 | | | | 250 | L/min |
| T1 | Toroidal | Water | 100 RPM | 50 | L/min |
| T2 | | | | 125 | L/min |
| T3 | | | | 250 | L/min |
| T4 | | | 150 RPM | 50 | L/min |
| T5 | | | | 125 | L/min |
| T6 | | | | 250 | L/min |
| T7 | | | 200 RPM | 50 | L/min |
| T8 | | | | 125 | L/min |
| T9 | | | | 250 | L/min |
| T10 | | Xanthan | 100 RPM | 50 | L/min |
| T11 | | Gum | | 125 | L/min |
| T12 | | | | 250 | L/min |
| T13 | | | 150 RPM | 50 | L/min |
| T14 | | | | 125 | L/min |
| T15 | | | | 250 | L/min |
| T16 | | | 200 RPM | 50 | L/min |
| T17 | | | | 125 | L/min |
| T18 | | | | 250 | L/min |

Results. The samples listed in Table 1 were tested under the corresponding conditions. For each sample, the volumetric mass transfer coefficient (kLa) was calculated, the average mixing time was calculated, and the average active power total was calculated, using standard measurement and calculation techniques for these metrics.

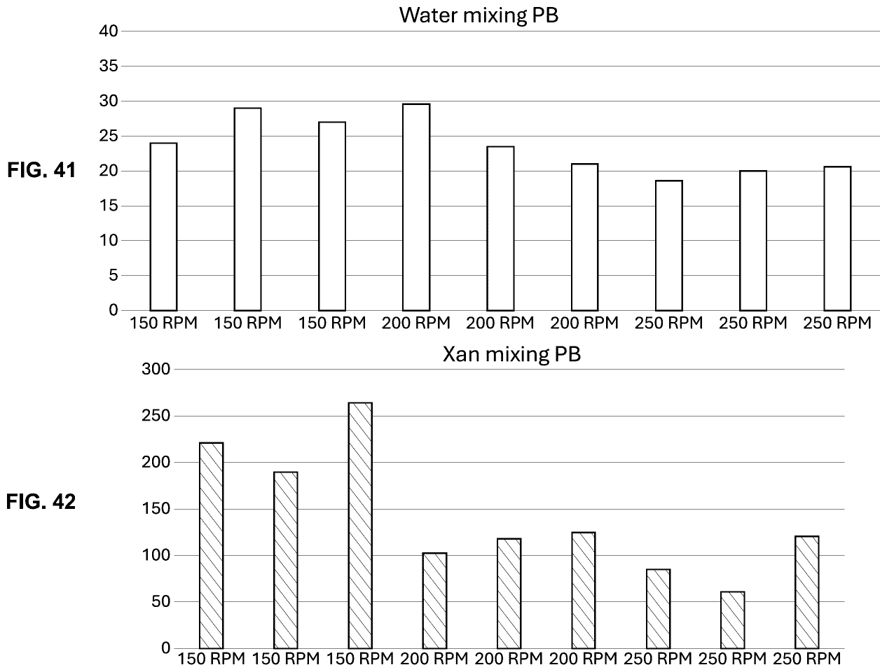

Mixing time is illustrated in FIGS. 41 and 42. Here, the average mixing time for samples using water (P1 to P9, R1 to R9, and T1 to T9 in the Table 1 above) are shown in FIG. 41 according to the RPM used. Here, the samples T1 to T9, using the toroidal impeller, had the best mixing time, regardless of RPM, compared to the pitch blade and Rushton samples.

Similarly, the average mixing time for samples using xanthan gum (P10 to P18, R10 to R18, and T10 to T18 in the Table 1 above) are shown in FIG. 42 according to the RPM used. Here, the samples T10 to T18, using the toroidal impeller, had the best mixing time, regardless of RPM, compared to the pitch blade and Rushton samples.

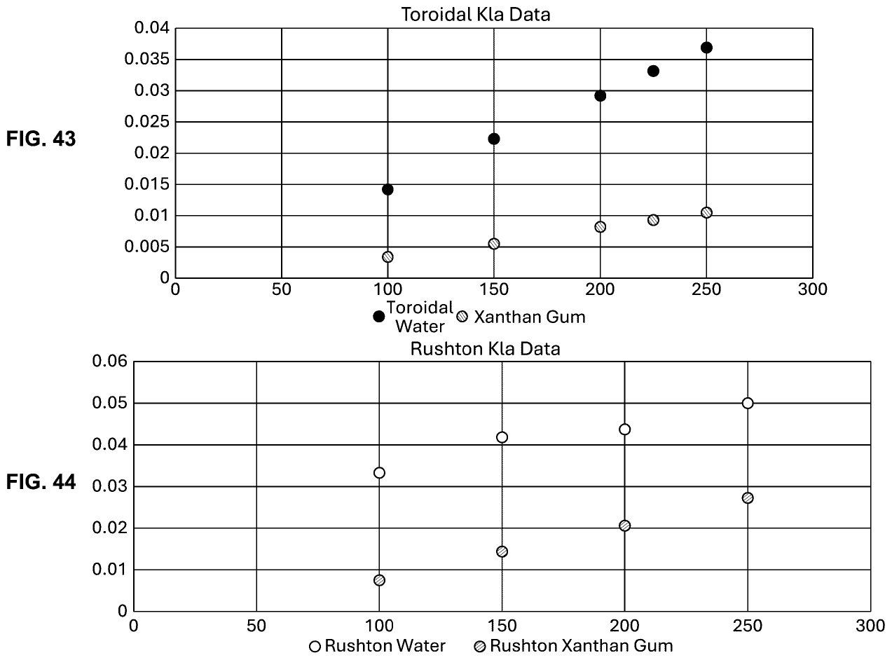

kLa data for the toroidal samples (T1 to T18) and for the Rushton samples (R1 to R18) are illustrated in FIGS. 43 and 44, respectively. In these plots, kLa is shown on the y-axis, while time is displayed on the x-axis. FIG. 43 depicts the kLa data for the toroidal samples T1 to T18, with the first series showing the water samples, and the second series showing the xanthan gum containing samples. FIG. 44 depicts the kLa data for the Rushton samples R1 to R18, with the first series showing the water samples, and the second series showing the xanthan gum containing samples. The water samples had a higher kLa overall, and the Rushton samples had a generally higher average kLa compared to the toroidal samples.

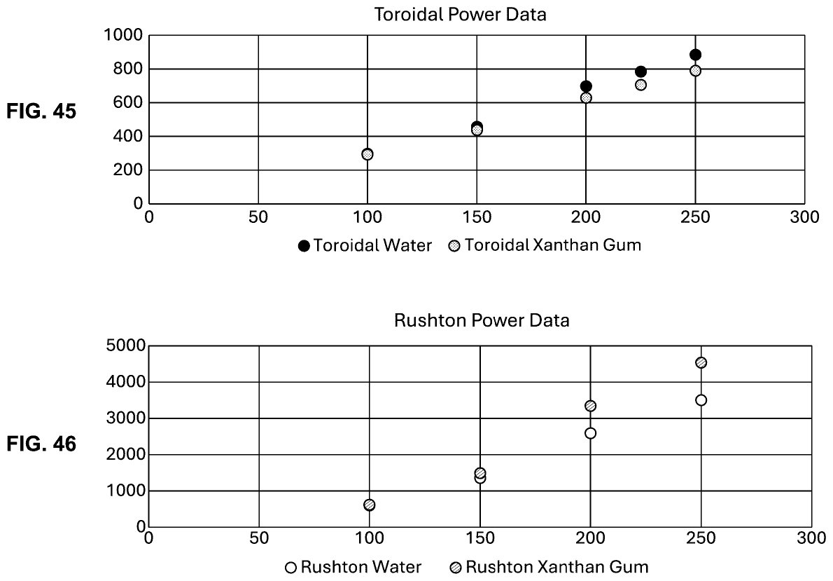

Active Power total for the toroidal samples (T1 to T18) and for the Rushton samples (R1 to R18) are illustrated in FIGS. 45 and 46, respectively. Again, these plots are separated out in to average power data (y-axis) for the toroidal samples and the Rushton samples. Overall, the toroidal impeller samples used less active power over the course of the testing.

### Example 2. Multiple Impeller Testing

In Example 2, a series of impellers were subject to oxygen transmission rate (OTR) testing, evaluated for kLa, and further tested for mixing time both with and without gas.

In each sample, three impellers were used in series in the same bioreactor chamber. The impellers were run simultaneously, equally spaced out within the bioreactor chamber. The testing was run under the same load for each test. Three equally placed sensors within the chamber were used for measuring impedance, which was converted to kLa.

The various impellers used included a Rushton type impeller (example in FIG. 28), a Marine type impeller (example in FIG. 29), a Toroidal 1 impeller (example in FIG. 5), a Toroidal 2 impeller (example in FIG. 23), and a Toroidal 3 impeller (example in FIGS. 21-26).

The batches that were run for Example 2 are summarized in the below Table:

TABLE 2
Example 2 samples

| Sample | Impeller 1 | Impeller 2 | Impeller 3 | Shaft Speed | Flow |
|---|---|---|---|---|---|
| No. | (Top) | (Middle) | (Bottom) | (RPM) | Direction |
| 2.01 | Rushton | Rushton | Rushton | 200 | Down |
| 2.02 | Toroidal 1 | Rushton | Rushton | 200 | Down |
| 2.03 | Toroidal 1 | Rushton | Rushton | 240 | Down |
| 2.04 | Toroidal 2 | Rushton | Rushton | 200 | Down |
| 2.05 | Toroidal 2 | Rushton | Rushton | 240 | Down |
| 2.06 | Toroidal 2 | Rushton | Rushton | 200 | Down |
| 2.07 | Toroidal 2 | Rushton | Rushton | 240 | Down |
| 2.08 | Toroidal 3 | Rushton | Rushton | 200 | Down |
| 2.09 | Toroidal 3 | Rushton | Rushton | 240 | Down |
| 2.10 | Marine | Rushton | Rushton | 200 | Down |
| 2.11 | Marine | Rushton | Rushton | 240 | Down |
| 2.12 | Toroidal 1 | Toroidal 1 | Toroidal 1 | 500 | Down |
| 2.13 | Toroidal 1 | Toroidal 1 | Toroidal 1 | 500 | Up |
| 2.14 | Toroidal 2 | Toroidal 2 | Toroidal 2 | 500 | Down |
| 2.15 | Toroidal 2 | Toroidal 2 | Toroidal 2 | 500 | Up |
| 2.16 | Toroidal 2 | Toroidal 2 | Toroidal 2 | 500 | Down |
| 2.17 | Toroidal 2 | Toroidal 2 | Toroidal 2 | 500 | Up |
| 2.18 | Toroidal 3 | Toroidal 3 | Toroidal 3 | 500 | Down |
| 2.19 | Toroidal 3 | Toroidal 3 | Toroidal 3 | 500 | Up |
| 2.20 | Toroidal 3 | Toroidal 3 | Toroidal 3 | 300 | Down |
| 2.21 | Marine | Marine | Marine | 500 | Down |
| 2.22 | Marine | Marine | Marine | 500 | Up |
| 2.23 | Toroidal 1 | Toroidal 1 | Rushton | 300 | Down |
| 2.24 | Toroidal 2 | Toroidal 2 | Rushton | 300 | Down |
| 2.25 | Toroidal 2 | Toroidal 2 | Rushton | 300 | Down |
| 2.26 | Toroidal 3 | Toroidal 3 | Rushton | 300 | Down |
| 2.27 | Marine | Marine | Rushton | 300 | Down |

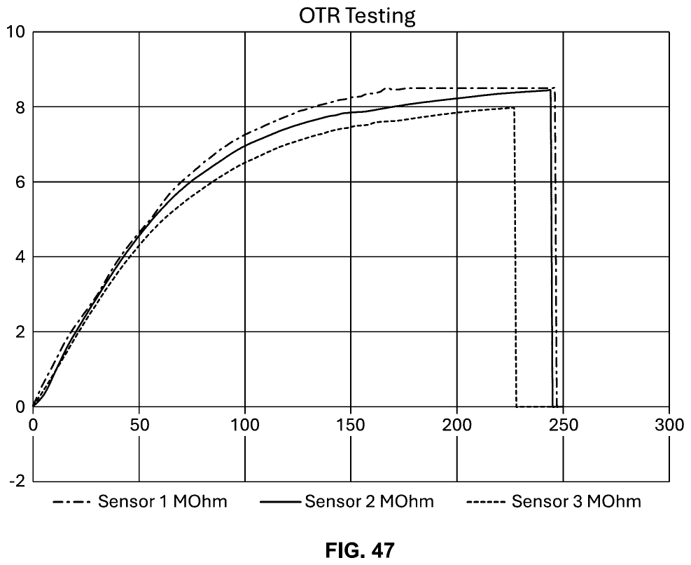

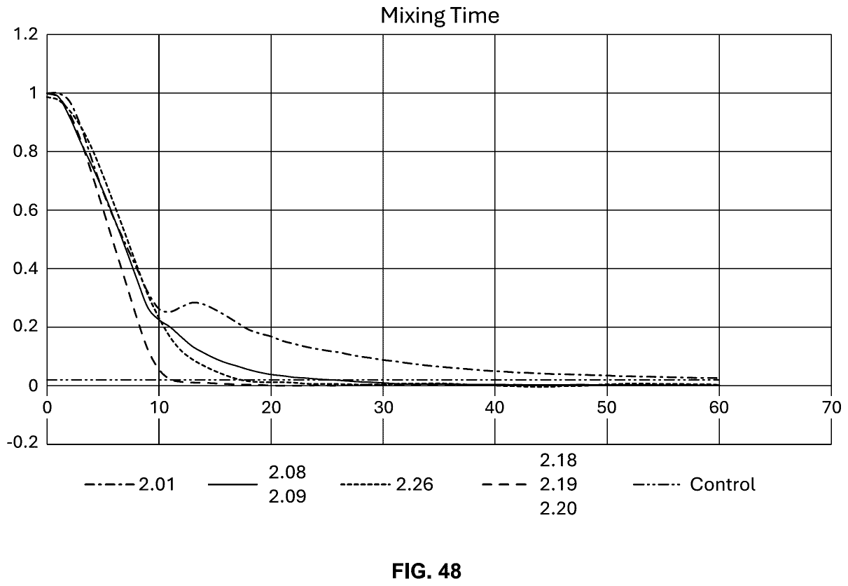

Results. The overall average OTR results are exhibited in FIG. 47. The max value was 8.5 mg/L, starting at 1.7 mg/L and stopping at 6.8 mg/L.

The average kLa values and mixing times are summarized for all the samples in the below Table:

TABLE 3
Example 2 results

| | | Avg. Bulk | Avg. Bulk |
|---|---|---|---|
| Sample | kLa | Mixing Rate (s) | Mixing Rate (s) |
| No. | (1/h) | (no gas) | (gas) |
| 2.01 | 90.00 | 37.33 | 35.33 |
| 2.02 | 79.27 | 32.33 | 29.33 |
| 2.03 | 78.44 | 24.67 | 24.00 |
| 2.04 | 72.20 | 29.00 | 32.00 |
| 2.05 | 77.83 | 24.67 | 24.67 |
| 2.06 | 68.69 | 27.00 | 29.33 |
| 2.07 | 75.81 | 23.33 | 26.33 |
| 2.08 | 82.42 | 27.67 | 28.67 |
| 2.09 | 85.99 | 22.33 | 23.00 |
| 2.10 | 76.87 | 33.33 | 28.33 |
| 2.11 | 78.17 | 25.33 | 27.00 |
| 2.12 | 62.61 | 13.67 | 15.33 |
| 2.13 | 60.61 | 11.33 | 12.00 |
| 2.14 | 73.56 | 14.33 | 15.67 |
| 2.15 | 72.17 | 14.00 | 13.00 |
| 2.16 | 65.91 | 16.33 | 18.00 |
| 2.17 | 70.15 | 18.33 | 18.33 |
| 2.18 | 101.67 | 13.00 | 17.33 |
| 2.19 | 92.66 | 14.33 | 19.33 |
| 2.20 | 67.86 | 17.00 | |
| 2.21 | 67.67 | 15.33 | 16.67 |
| 2.22 | 68.82 | 16.67 | 15.67 |
| 2.23 | 75.30 | 17.67 | 18.00 |
| 2.24 | 80.56 | 19.67 | 18.33 |
| 2.25 | 76.60 | 16.33 | 19.67 |
| 2.26 | 86.38 | 16.00 | 22.67 |
| 2.27 | 75.00 | 18.67 | 23.00 |

The mixing time results are also summarized in FIG. 48, where several sample averages are exhibited: 2.01 (three Rushton type impellers), the average of 2.08/2.09 (Toroidal 3 and two Rushton type impellers), 2.26 (two Toroidal 3 and one Rushton type impeller), the average of 2.18/2.19/2.20 (three Toroidal 3 impellers), and a Control which was homogenous. Overall, the mixing time it took to get to a homogenous mixture (e.g., to meet the ‘Control’ curve) was quickest in the 2.18, 2.19, 2.20 samples using three toroidal impellers.

### Example 3. Fermentation Testing (10 L Batches)

In Example 3, several 10 L batches were tested with bioreactors having toroidal impellers. Here, the same size bioreactor compartment was run with the same load. The impellers used in Example 3 were toroidal impellers as shown in FIG. 23.

In each of the Example 3 tests, a bacteria of the strain E. coli producing green fluorescent protein (GFP) was used. For the fermentation process, the following steps were used: cyrovials or plate colonies were cultured to seed flasks, cultured up to seed tanks, and then cultured to production tanks.

In Example 3, the final production tanks used were 10 L in size. They were prepared from seed flasks as shown in the below Table:

TABLE 4
Fermentation testing summary

| | Seed flasks | | Production tank | | |
|---|---|---|---|---|---|
| | 0.5 L | | 10 L | | |
| | | Seed | | Seed | |
| | Working | Inoculum | Working | Inoculum | |
| | volume | volume | volume | volume | |
| 3A | 500 mL x2 | 0.5 mL | 8 L | 0.4 | L |
| 3B | 100 mL x2 | 0.1 mL x2 | | 0.04 | L |

| | | |
|---|---|---|
| 3C | 50 mL x2 | Colonies x2 |

| | | |
|---|---|---|
| 3D | Cryovials | 2 mL cryovials |

The fermentation medium used in tests 3A to 3D is summarized in the below Table.

TABLE 5
Fermentation testing medium

| | Seed | Production tank | Feeding medium |
|---|---|---|---|
| | Medium | culture 10 L | in fed-batch |
| Carbon | Glycerol | Glycerol | Glycerol |
| Nitrogen | Trypton | Soy Peptone | Soy Peptone |
| | YE (NuCel 532) | Yeast Extract | Yeast Extract |
| | | (Nucel 532 MG) | (Nucel 532 MG) |
| Salts | Potassium | Potassium | — |
| | phosphate dibasic | phosphate dibasic | |
| | k2HPO4 | k2HPO4 | |
| | Potassium | Potassium | |
| | phosphate | phosphate | |
| | monobasic | monobasic | |
| | kH2PO4 | kH2PO4 | |
| | | Sodium citrate | |
| | | Ammonium | |
| | | chloride | |

The mediums also included trace minerals and antifoam as needed. Acid and base additions were additionally used to adjust pH as desired. For each test 3A to 3D, fed-batch was added to the bioreactor at OD600 @20-25 at a feed volume of 2 L.

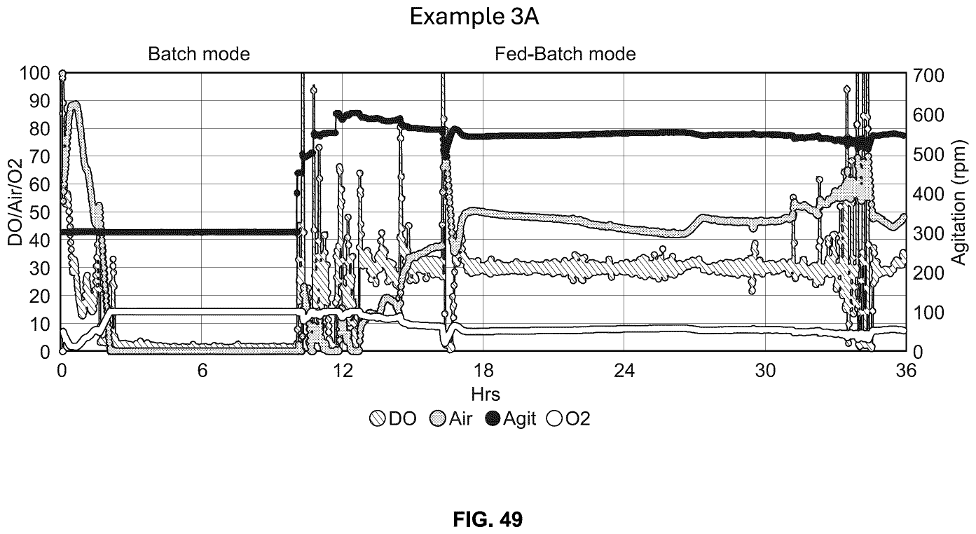

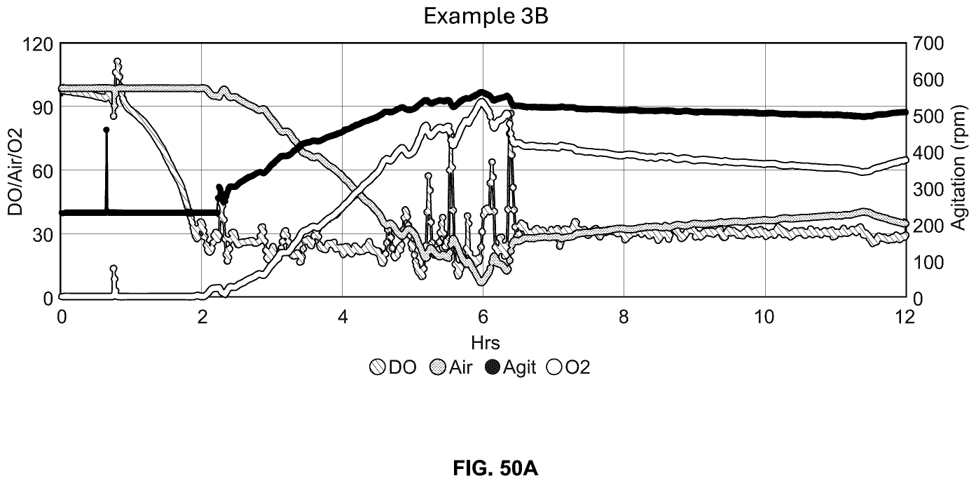

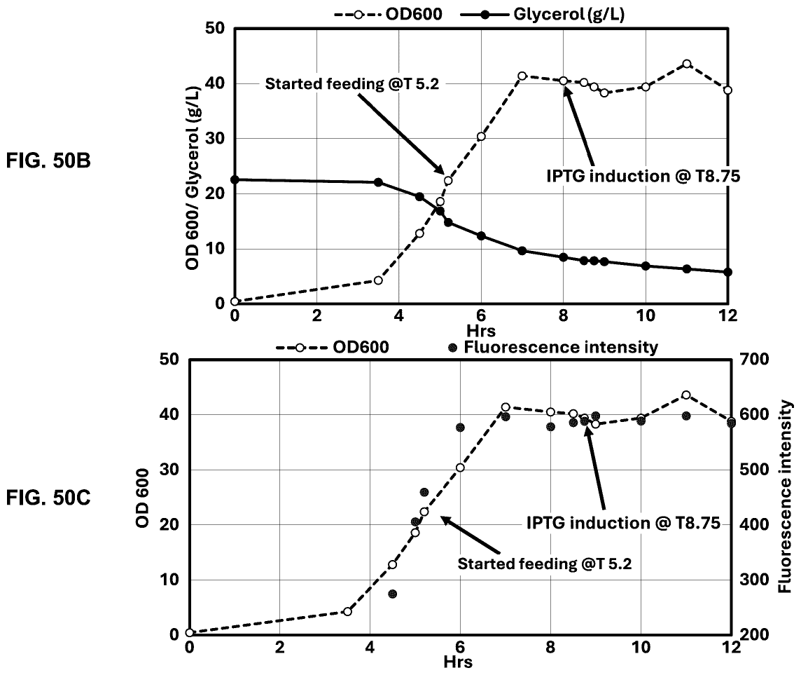

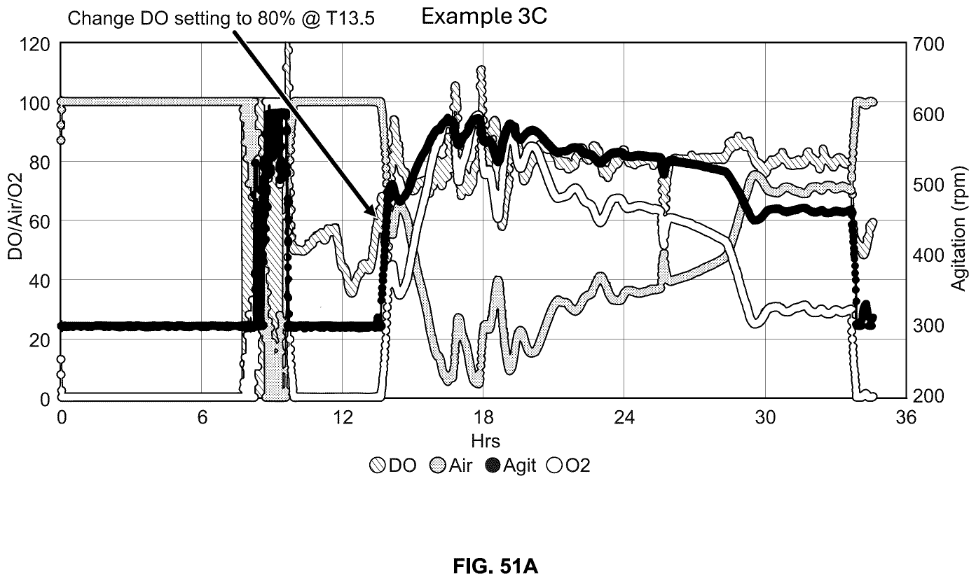

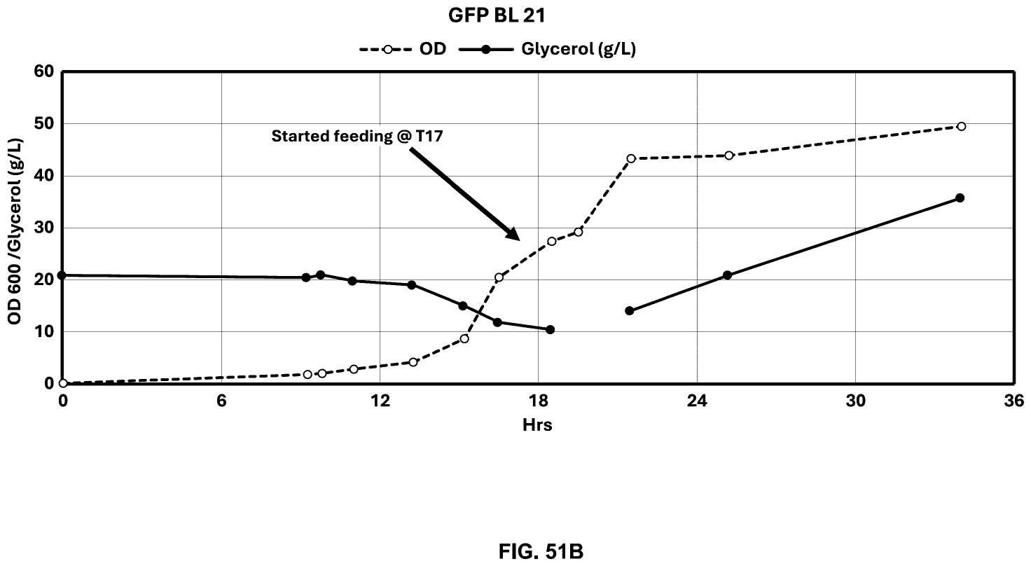

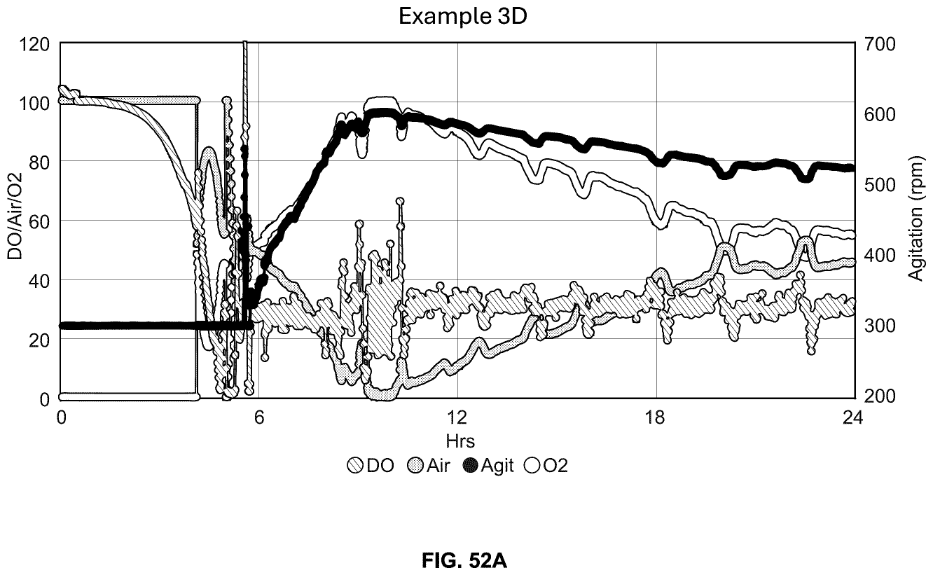

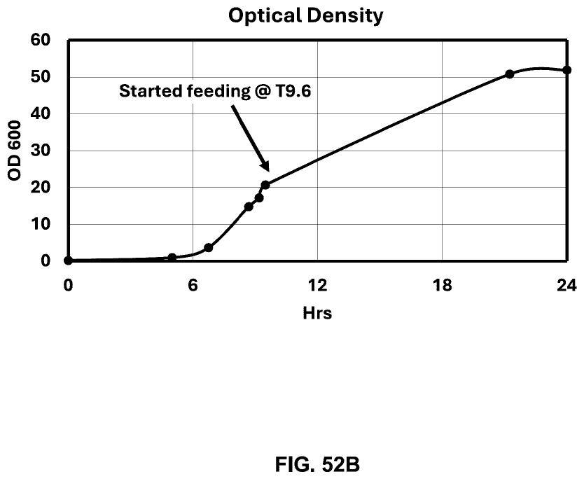

Results. Test 3A results are shown in FIG. 49. Test 3B results are shown in FIGS. 50A-50C. Test 3C results are shown in FIGS. 51A-51B. Test 3D results are shown in FIGS. 52A-52B.

Test 3A was run in a 10 L production tank with a seed inoculum of 0.5%, at a temperature of 30° C., an air flow of 0.5 vvm, a dissolved oxygen (DO) of 30%, a cascade agitation of 300 to 600 rpm, pure oxygen, and a pH of 7. FIG. 49 depicts the overall fermentation trends for Test 3A, including dissolved oxygen (DO), air, agitation (Agit), and oxygen (O2).

Test 3B was run in a 10 L production tank with a seed inoculum of 0.5%, at a temperature of 37° C., an air flow of 0.5 vvm, a dissolved oxygen (DO) of 30%, a cascade agitation of 300 to 600 rpm, pure oxygen, and a pH of 7. FIG. 50A depicts the overall fermentation trends for Test 3B, including dissolved oxygen (DO), air, agitation (Agit), and oxygen (O2).

FIG. 50B and FIG. 50C depict the glycerol fluorescence measured during Test 3B. The OD600 (optical density at 600 nm) can be seen increasing when batch feeding began at 5.2 hours, when the glycerol begins to reduce. The IPTG (Isopropyl β-D-thiogalactopyranoside) induction, which acts as an inducer of the lac operon in E. coli, can be seen around 8.75 hours.

Test 3C was run in a 10 L production tank with a seed inoculum of 0.5%, at a temperature of 28° C. overnight and 30° C. thereafter, an air flow of 0.5 vvm, a dissolved oxygen (DO) of 30%, a cascade agitation of 300 to 600 rpm, pure oxygen, and a pH of 7. FIG. 51A depicts the overall fermentation trends for Test 3C, including dissolved oxygen (DO-1), air (1), oxygen (O2 (2)), and agitation (Agit) during this test. Shown in FIG. 51B, when the feeding began at time 17 hours, there was a marked increase in OD.

Test 3D was run in a 10 L production tank with a seed inoculum of 0.5%, at a temperature of 37° C., an air flow of 0.5 vvm, a dissolved oxygen (DO) of 30%, a cascade agitation of 300 to 600 rpm, pure oxygen, and a pH of 7. FIG. 52A depicts the overall fermentation trends for Test 3C, including oxygen (O2), air, dissolved oxygen (DO), and agitation (Agit). The optical density in Test 3D is depicted in FIG. 52B. The batch fed began at about 9.6 hours.

### Example 4. Fermentation Testing (500 L Batches)

In Example 4, several 500 L batches were tested with bioreactors having toroidal impellers. Here, the same size bioreactor compartment was run with the same load. The impellers used in Example 4 were toroidal impellers as shown in FIG. 23.

In each of the Example 4 tests, a bacteria of the strain E. coli GFP was used. For the fermentation process, the following steps were used: cyrovials or plate colonies were cultured to seed flasks, cultured up to seed tanks, and then cultured to production tanks.

In Example 4, the final production tanks used were 500 L in size. They were prepared from seed flasks as shown in the below Table:

TABLE 6
Fermentation testing summary

| | |
|---|---|
| Seed flasks | Production tank |

| | | Seed | | Seed | |
|---|---|---|---|---|---|
| | Working | Inoculum | Working | Inoculum | |
| | volume | volume | volume | volume | |
| 4A | 500 mL x3 | 0.5 mL x3 | 400 L | 20 | L |
| 4B | 500 mL x4 | 0.5 mL x4 | | 2 | L |

The fermentation medium used in tests 4A to 4B is summarized in the below Table.

TABLE 7
Fermentation testing medium

| | Seed | Production tank | Feeding medium |
|---|---|---|---|
| | Medium | culture | in fed-batch |
| Carbon | Glycerol | Glycerol | Glycerol |
| Nitrogen | Trypton | Soy Peptone | Soy Peptone |
| | YE (NuCel 532) | Yeast Extract | Yeast Extract |
| | | (Nucel 532 MG) | (Nucel 532 MG) |
| Salts | Potassium | Potassium | — |
| | phosphate dibasic | phosphate dibasic | |
| | k2HPO4 | k2HPO4 | |
| | Potassium | Potassium | |
| | phosphate | phosphate | |
| | monobasic | monobasic | |
| | kH2PO4 | kH2PO4 | |
| | | Sodium citrate | |
| | | Ammonium | |
| | | chloride | |

The mediums also included trace minerals and antifoam as needed. Acid and base additions were additionally used to adjust pH as desired. For each test 4A and 4B, fed-batch of glycerol was added to the bioreactor at OD600 @20-25 at a feed volume of 100 L with a fixed feed rate of 80 mL/min.

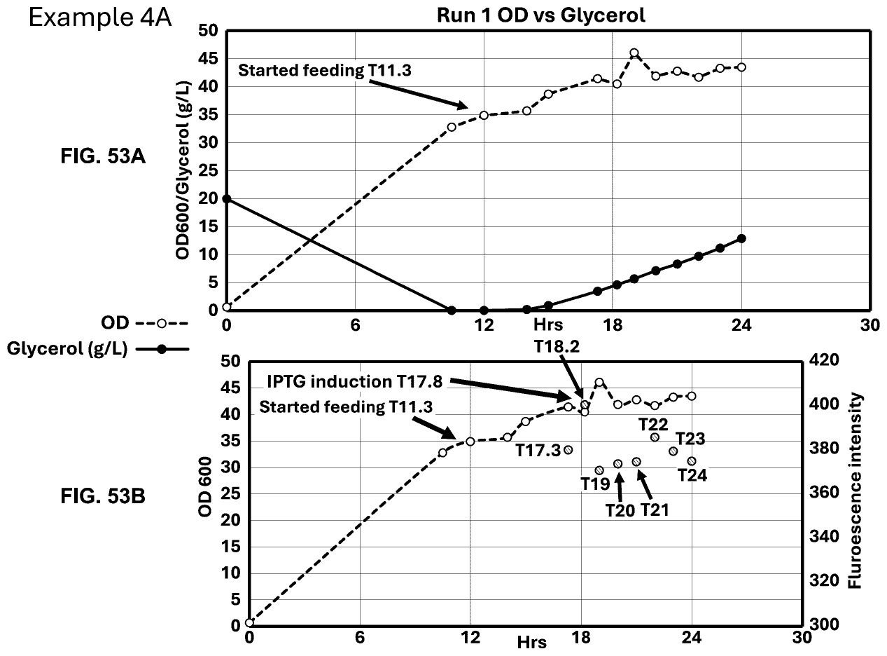

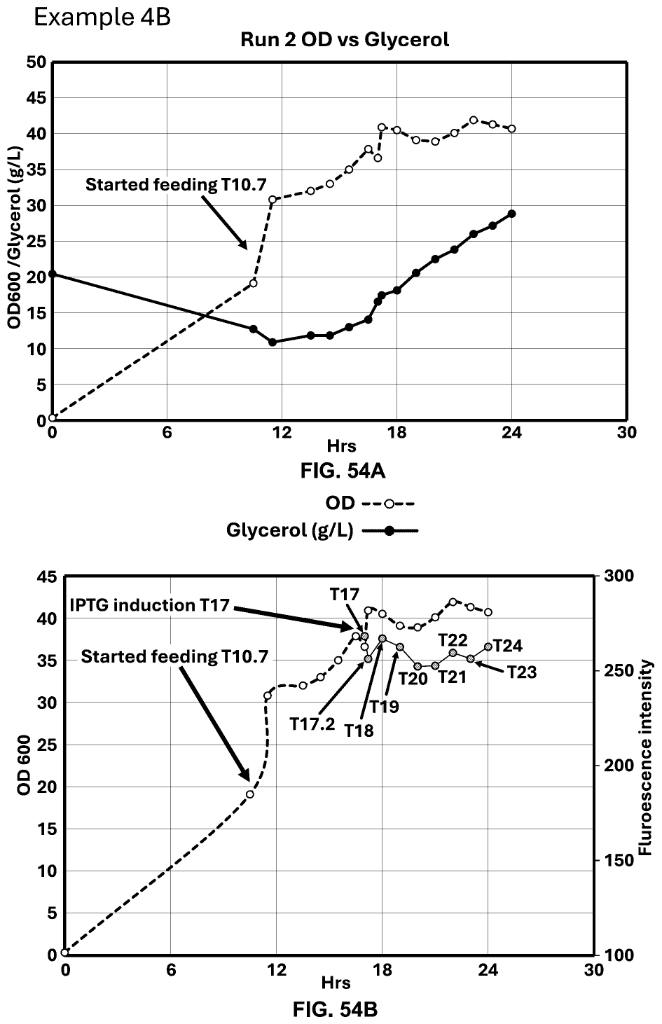

Results. Test 4A results are shown in FIGS. 53A to 53B. Test 4B results are shown in FIGS. 54A to 54B.

Test 4A was run in a 500 L production tank with a seed inoculum of 0.5%, at a temperature of 30° C. (with an increase to 37° C. overnight and reduction back to 30° C.), an air flow of 0.5 vvm, an agitation of 300 rpm, and a pH of 7. In Test 4A, the batch feed was started at 11.3 hours, and IPTG induction occurred around 17.8 hours, as exhibited in FIGS. 53A and 53B.

Test 4B was run in a 500 L production tank with a seed inoculum of 0.5%, at a temperature of 30° C., an air flow of 0.5 vvm, an agitation of 300 rpm, pure oxygen, and a pH of 7. In Test 4B, the batch feed was started at 10.7 hours, while the IPTG induction occurred at 17 hours, as exhibited in FIGS. 54A and 54B.

### ADDITIONAL ASPECTS

The present disclosure includes various aspects that may be claimed in the future. The following aspects are intended to highlight certain features without limiting the scope of protection. It should be understood that any of the following aspects may be claimed in a patent application claiming priority to the present disclosure, either alone or in combination with other aspects. Further, the following aspects may be modified or combined in any suitable manner apparent to one skilled in the art in light of the teachings herein. Features which are described in the context of separate aspects and embodiments of the disclosure may be used together and/or be interchangeable. Similarly, features described in the context of a single embodiment may also be provided separately or in any suitable subcombination. The aspects are numbered for convenience only and should not be construed as requiring a particular order or limiting the scope of what may be claimed. In various aspects, mixing tanks, bioreactors, and methods are provided that include features and components as described in one or more of the following aspects. The aspects may be combined or modified in ways apparent to those skilled in the art based on the teachings herein. While specific materials, dimensions, and configurations are described for certain aspects, these are exemplary only and other materials, dimensions and configurations may be used within the scope of the disclosure.

Aspect 1. A mixing tank arrangement comprising: a) a vessel defining an interior volume; b) a rotatable shaft assembly extending within the interior volume; and c) one or more impellers mounted onto the shaft and disposed within the interior volume, the one or more impellers including one or more toroidal impellers.

Aspect 2. The mixing tank arrangement of Aspect 1, or any of Aspects 3 to 52, wherein the one or more toroidal impellers includes at least one blade member having first and second blade ends, and wherein at least one of the first and second blade ends adjoins an outer surface of a hub.

Aspect 3. The mixing tank arrangement of Aspect 2, or any of Aspects 1 and 4 to 52, wherein both the first and second blade ends of the at least one blade member adjoin the outer surface of the hub.

Aspect 4. The mixing tank arrangement of Aspect 2, or any of Aspects 1, 3, and 5 to 52, wherein an inner side surface of the at least one blade member defines a radially bounded passageway extending along a second axis disposed at an oblique angle to a longitudinal axis of the one or more toroidal impellers.

Aspect 5. The mixing tank arrangement of Aspect 1, or any of Aspects 2 to 4 and 6 to 52, wherein the one or more toroidal impellers includes at least two blade members.

Aspect 6. The mixing tank arrangement of Aspect 2, or any of Aspects 1, 3 to 5, and 7 to 52, wherein none of the blade members contacts another of the blade members.

Aspect 7. The mixing tank arrangement of Aspect 6, or any of Aspects 1 to 5 and 8 to 52, wherein the first end of the at least one blade member is axially separated from the second end of the at least one blade member.

Aspect 8. The mixing tank arrangement of Aspect 1, or any of Aspects 2 to 7 and 9 to 52, wherein the one or more impellers includes at least one impeller that is not a toroidal impeller.

Aspect 9. The mixing tank arrangement of Aspect 1, or any of Aspects 2 to 8 and 10 to 52, wherein the shaft assembly is supporting by a first bearing or bushing assembly and a second bearing or bushing assembly, and wherein the one or more toroidal impellers is located axially between the first and second bearing or bushing assemblies.

Aspect 10. A bioreactor comprising: a vessel defining an interior volume; a motor; a rotatable shaft assembly connected to the motor and extending within the interior volume along a longitudinal axis; one or more impellers mounted onto the shaft assembly and disposed within the interior volume, the one or more impellers including one or more toroidal impellers.

Aspect 11. The bioreactor of Aspect 10, or any of Aspects 1 to 9 and 12 to 52, wherein the motor, during operation, rotates the shaft assembly and the one or more toroidal impellers at a maximum RPM of about 1,500.

Aspect 12. The bioreactor of Aspect 10, or any of Aspects 1 to 9, 11, and 13 to 52, wherein the one or more toroidal impellers includes a plurality of blade members each having a first end and a second end, wherein at least one of the first and second blade ends of each of the plurality of blade members adjoins an outer surface of a hub.

Aspect 13. The bioreactor of Aspect 12, or any of Aspects 1 to 11 and 14 to 52, wherein both of the first and second blade ends of each of the plurality of blade members adjoins the outer surface of the hub.

Aspect 14. The bioreactor of any of Aspects 12 to 13, or any of Aspects 1 to 11 and 15 to 52, wherein an inner side surface of each of the plurality of blade members defines a radially bounded passageway.

Aspect 15. The bioreactor of any of Aspects 10 to 14, or any of Aspects 1 to 9 and 16 to 52, wherein the one or more toroidal impellers includes at least three blade members.

Aspect 16. The bioreactor of any of Aspects 12 to 15, or any of Aspects 1 to 11 and 17 to 52, wherein none of the plurality of blade members contacts another of the plurality of blade members.

Aspect 17. The bioreactor of Aspect 12, or any of Aspects 1 to 11, 13 to 16, and 18 to 52, wherein the first end of each of the plurality of blade members is axially separated from the second end of each of the plurality of blade members.

Aspect 18. The bioreactor of any of Aspects 10 to 17, or any of Aspects 1 to 9 and 19 to 52, wherein the one or more impellers includes an impeller that is not a toroidal impeller.

Aspect 19. The bioreactor of any of Aspects 10 to 18, or any of Aspects 1 to 9 and 20 to 52, wherein the shaft assembly is supported by a first bearing or bushing assembly and a second bearing or bushing assembly, and wherein the one or more toroidal impellers are located axially between the first and second bearing or bushing assemblies.

Aspect 20. The bioreactor of Aspect 1, or any of Aspects 2 to 19 and 21 to 52, wherein the one or more impellers includes at least one Rushton type impeller.

Aspect 21. The bioreactor of Aspect 20, or any of Aspects 1 to 19 and 22 to 52, wherein the one or more impellers includes two Rushton type impellers and a single toroidal impeller.

Aspect 22. The bioreactor of Aspect 21, or any of Aspects 1 to 20 and 23 to 52, wherein the single toroidal impeller is located axially between the two Rushton type impellers.

Aspect 23. The bioreactor of Aspect 21, or any of Aspects 1 to 20, 22, and 24 to 52, wherein the single toroidal impeller is located axially above the two Rushton impellers.

Aspect 24. The bioreactor of Aspect 10, or any of Aspects 1 to 9 and 11 to 23 and 25 to 52, wherein the shaft assembly is rotated such that the one or more impellers induces an upward fluid flow within the interior volume.

Aspect 25. The bioreactor of Aspect 24, or any of Aspects 1 to 23 and 26 to 52, wherein the one or more impellers includes only toroidal impellers.

Aspect 26. A single use bioreactor comprising: a flexible bag defining an interior volume; a rotatable shaft assembly extending within the interior volume of the flexible bag; and one or more impellers mounted onto the shaft assembly and disposed within the interior volume, the one or more impellers including one or more toroidal impellers.

Aspect 27. The single use bioreactor of Aspect 26, or any of Aspects 1 to 25 and 28 to 52, wherein the one or more impellers includes an impeller that is not a toroidal impeller.

Aspect 28. The single use bioreactor of Aspect 26, or any of Aspects 1 to 25, 27, and 29 to 52, wherein the one or more impellers, shaft assembly, and bearings are disposable.

Aspect 29. The single use bioreactor of any of Aspects 26 to 28, or any of Aspects 1 to 25 and 30 to 52, wherein the one or more impellers includes at least one Rushton type impeller.

Aspect 30. A bioreactor comprising: a vessel defining an interior volume; a motor; a rotatable shaft assembly connected to the motor and extending within the interior volume along a longitudinal axis; one or more impellers mounted onto the shaft and disposed within the interior volume, the one or more impellers including one or more toroidal impellers, wherein at least one of the one or more impellers includes surface openings for delivering a gaseous fluid to the interior volume.

Aspect 31. The bioreactor of Aspect 30, or any of Aspects 1 to 29 and 32 to 52, wherein at least some of the surface openings are defined on blades of the at least one impeller.

Aspect 32. The bioreactor of Aspect 31, or any of Aspects 1 to 30 and 33 to 52, wherein at least some of the surface openings are provided on a trailing face of the blades.

Aspect 33. The bioreactor of Aspect 31, or any of Aspects 1 to 30, 32, and 34 to 52, wherein at least some of the surface openings are provided on a leading face of the blades.

Aspect 34. The bioreactor of Aspect 30, or any of Aspects 1 to 29 and 31 to 33 and 35 to 52, wherein at least some of the surface openings are provided on a central hub portion of the at least one impeller.

Aspect 35. The bioreactor of Aspect 30, or any of Aspects 1 to 29 and 31 to 34 and 36 to 52, wherein at least some of the surface openings have a size of between 0.15 and 5.0 mm.

Aspect 36. A method of fermentation, the method comprising: providing a bioreactor vessel with one or more toroidal impellers mounted on a rotatable shaft; introducing a fermentation medium into the vessel and inoculating the fermentation medium; rotating the shaft and toroidal impellers; and introducing a gaseous fluid into the vessel to induce fermentation therein.

Aspect 37. The method of Aspect 36, or any of Aspects 1 to 35 and 38 to 52, wherein rotating the shaft is done at a speed between 100 and 1500 RPM.

Aspect 38. The method of Aspect 35, or any of Aspects 1 to 37 and 39 to 52, wherein rotating the shaft is done at a speed between 300 and 600 RPM.

Aspect 39. The method of Aspect 36, or any of Aspects 1 to 35 and 37 to 38 and 40 to 52, wherein introducing a gaseous fluid into the vessel is done at a flow rate between 50 L/min and 250 L/min and/or at a flow rate between 0.1 and 1.0 vessel volumes per minute.

Aspect 40. The method of Aspect 36, or any of Aspects 1 to 35 and 37 to 39 and 41 to 52, wherein introducing a gaseous fluid comprises flowing air into the vessel.

Aspect 41. The method of Aspect 40, or any of Aspects 1 to 39 and 42 to 52, wherein the air is provided at an air flow of 0.5 vvm.

Aspect 42. The method of Aspect 36, or any of Aspects 1 to 35 and 37 to 41 and 43 to 52, wherein inoculating the medium comprises applying one or more bacteria to the medium.

Aspect 43. The method of Aspect 42, or any of Aspects 1 to 41 and 44 to 52, further comprising inducing protein expression in the one or more bacteria.

Aspect 44. The method of Aspect 36, or any of Aspects 1 to 35 and 37 to 43 and 45 to 52, further comprising adding fed-batch medium to the vessel at a threshold optical density.

Aspect 45. The method of Aspect 36, or any of Aspects 1 to 35 and 37 to 44 and 46 to 52, wherein the fermentation medium comprises glycerol or glucose as a carbon source.

Aspect 46. The method of Aspect 36, or any of Aspects 1 to 35 and 37 to 45 and 47 to 52, wherein the method is done at a temperature of 28 to 37° C.

Aspect 47. The method of Aspect 36, or any of Aspects 1 to 35 and 37 to 46 and 48 to 52, wherein the method is done at a neutral pH.

Aspect 48. A method of enhancing mass transfer in a bioreactor comprising: providing multiple impellers including at least one toroidal impeller; operating the impellers simultaneously; and achieving volumetric mass transfer coefficient (kLa) values between 60-102 h⁻¹.

Aspect 49. The method of Aspect 48, or any of Aspects 1 to 47 and 50 to 52, wherein operating the impellers is done at a speed of up to 500 RPM.

Aspect 50. The method of Aspect 48, or any of Aspects 1 to 47, 49, and 51 to 52, further comprising obtaining improved mixing times of 10 to 20 seconds compared to configurations not including a toroidal impeller.

Aspect 51. A method of culturing cells in a bioreactor, the method comprising: providing a liquid with gas bubbles in the bioreactor; agitating the liquid to divide the gas bubbles with at least one toroidal impeller in the bioreactor; delivering the liquid with the divided gas bubbles to a cell culture bed.

Aspect 52. A method of low shear mixing a culture medium in a bioreactor, the method comprising: providing a toroidal impeller in the bioreactor, the toroidal impeller having blade members defining radially bounded passageways; filling the bioreactor with the culture medium, the culture medium including one or more microorganisms; rotating the impeller to create axial flow with reduced turbulent eddies; mixing while maintaining shear stress below levels that damage the microorganisms; and obtaining homogenous distribution of nutrients, gases, and temperature throughout the culture medium.

The present invention has now been described with reference to several embodiments thereof. The entire disclosure of any patent or patent application identified herein is hereby incorporated by reference. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein, but only by the structures described by the language of the claims and the equivalents of those structures.

What is claimed is:

1. A mixing tank arrangement comprising:
a) a vessel defining an interior volume;
b) a rotatable shaft assembly extending within the interior volume; and
c) one or more impellers mounted onto the shaft assembly and disposed within the interior volume, the one or more impellers including one or more toroidal impellers.

2. The mixing tank arrangement of claim 1, wherein the one or more toroidal impellers includes at least one blade member having first and second blade ends, and wherein at least one of the first and second blade ends adjoins an outer surface of a hub.

3. The mixing tank arrangement of claim 2, wherein both of the first and second blade ends of the at least one blade member adjoin the outer surface of the hub.

4. The mixing tank arrangement of claim 2, wherein an inner side surface of the at least one blade member defines a radially bounded passageway extending along a second axis disposed at an oblique angle to a longitudinal axis of the one or more toroidal impellers.

5. The mixing tank arrangement of claim 1, wherein the one or more toroidal impellers includes at least two blade members.

6. The mixing tank arrangement of claim 2, wherein none of the blade members contacts another of the blade members.

7. The mixing tank arrangement of claim 6, wherein the first end of the at least one blade member is axially separated from the second end of the at least one blade member.

8. The mixing tank arrangement of claim 1, wherein the one or more impellers includes at least one impeller that is not a toroidal impeller.

9. The mixing tank arrangement of claim 1, wherein the shaft assembly is supported by a first bearing or bushing assembly and a second bearing or bushing assembly, and wherein the one or more toroidal impellers are located axially between the first and second bearing or bushing assemblies.

10. A bioreactor comprising:
a) a vessel defining an interior volume;
b) a motor;
c) a rotatable shaft assembly connected to the motor and extending within the interior volume along a longitudinal axis;
d) one or more impellers mounted onto the shaft assembly and disposed within the interior volume, the one or more impellers including one or more toroidal impellers.

11. The bioreactor of claim 10, wherein the motor, during operation, rotates the shaft assembly and the one or more toroidal impellers at a maximum RPM of about 1,500.

12. The bioreactor of claim 10, wherein the one or more toroidal impellers includes a plurality of blade members each having a first end and a second end, wherein at least one of the first and second blade ends of each of the plurality of blade members adjoins an outer surface of a hub.

13. The bioreactor of claim 12, wherein both of the first and second blade ends of each of the plurality of blade members adjoins the outer surface of the hub.

14. The bioreactor of claim 12, wherein an inner side surface of each of the plurality of blade members defines a radially bounded passageway.

15. The bioreactor of claim 12, wherein the one or more toroidal impellers includes at least three blade members.

16. The bioreactor of claim 12, wherein none of the plurality of blade members contacts another of the plurality of blade members.

17. The bioreactor of claim 12, wherein the first end of each of the plurality of blade members is axially separated from the second end of each of the plurality of blade members.

18. The bioreactor of claim 10, wherein the one or more impellers includes at least one impeller that is not a toroidal impeller.

19. The bioreactor of claim 10, wherein the shaft assembly is supported by a first bearing or bushing assembly and a second bearing or bushing assembly, and wherein the one or more toroidal impellers are located axially between the first and second bearing or bushing assemblies.

20. The bioreactor of claim 1, wherein the one or more impellers includes at least one Rushton type impeller.
